(19) United States
(12) Patent Application Publication
(10) Pub. No.: US 2023/0033234 A1
(43) Pub. Date: Feb. 2, 2023

(54) BONE IMPACTOR SYSTEMS AND PROCESSES FOR USING SAME

(71) Applicant: TETRAX LLC, Atlanta, GA (US)

(72) Inventors: Nitin Agarwal, Flemington, NJ (US); Jacob Rahul Rajiv Joseph, Ann Arbor, MI (US); Stephen N. Housley, Atlanta, GA (US); David Wu, Atlanta, GA (US)

(21) Appl. No.: 17/716,656

(22) Filed: Apr. 8, 2022

Related U.S. Application Data
(63) Continuation of application No. PCT/US22/23990, filed on Apr. 8, 2022.
(60) Provisional application No. 63/203,830, filed on Aug. 2, 2021.

(51) Int. Cl. A61F 2/46; A61B 90/00

(52) U.S. Cl. CPC A61F 2/4644; A61B 90/39; A61F 2002/2835

(57) ABSTRACT

A bone graft generation and delivery process can include inserting bone material into an impactor system. The process can include processing, via the impactor system, the bone material into bone graft material. Processing the bone material can include milling the bone material via a first rotational element of the impactor system. Processing the bone material can include cutting the bone material via a second rotational element of the impactor system. Processing the material can include filtering the cut and milled bone material to isolate bone graft material. The process can include delivering, via the impactor system, the bone graft material to a target site.

### CROSS REFERENCE TO RELATED APPLICATION

This application is a continuation of International Application PCT/US22/23990, filed Apr. 8, 2022, entitled “BONE IMPACTOR SYSTEMS AND PROCESSES FOR USING SAME,” which claims the benefit of and priority to U.S. Application Ser. No. 63/203,830, filed Aug. 2, 2021, entitled “AN INTEGRATED, PORTABLE POWERED SURGICAL BONE MILL AND BONE GRAFT DELIVERY SYSTEM AND METHODS FOR USING SAME,” the disclosures of which are incorporated herein by reference in their entireties.

### TECHNICAL FIELD

The present systems and processes relate generally to orthopedic and/or neurologic surgical devices and processes.

### BACKGROUND

Bone grafts help treat various orthopedic and neurologic problems, e.g. fusing a joint, decompressing a spinal nerve, or repairing a fracture. In a bone grafting procedure, a surgeon manually places bone grafts into an area in a patient's body to provide a type of scaffold for bone growth and repair.

This procedure typically requires two broad steps. First, generation of bone graft material. Second, implantation of bone graft at the surgical site. Unfortunately, these processes are time consuming, inefficient, and typically results in inadequate delivery of material.

Bone grafts are typically generated de novo, intraoperatively from autogenous (from the patient's own body) or allogeneic (from a donor). For example, during a procedure to fuse spinal segments, a surgeon removes a portion of bone from a different location in the same patient (autogenic). The surgeon grinds the bone using a hand-powered rasp or bone mill. This is a relatively long and strenuous operation often with mixed results depending on the bone and the rasp and/or bone mill. The surgeon then manually places the graft into the surgical location, by hand. These manually intensive steps necessarily result in increased intraoperative time and increased risk of blood loss. Further, due to inconsistent, imprecise, and inadequate filling of target location with bone graft, bones may not optimally fuse leading to worse (less stable) surgical outcomes.

Therefore, there is a long-felt but unresolved need for an improved system and process for generating and delivering bone graft material.

### BRIEF SUMMARY OF THE DISCLOSURE

Briefly described, and according to one embodiment, aspects of the present disclosure generally relate to systems and processes for generating and delivering bone graft material.

In various embodiments, the present application relates to the generation of bone graft material and delivery of bone graft material to a target site. In at least one embodiment, the systems and methods described herein allow for the simultaneous generation and navigated delivery of bone graft material to a desired location in an open or minimally invasive procedure via a portable powered surgical bone mill and delivery system. In one or more embodiments, the systems and methods described herein may overcome disadvantages of conventional systems and methods for bone graft generation and delivery. For example, past approaches to preparing bone graft material may rely on manually powered devices and techniques that undesirably fatigue the user. In contrast, the present bone impactor system may improve upon past approaches by providing an automated electromechanical means for preparing and delivering bone graft material.

In various embodiments, the present disclosure shows and describes a portable powered surgical bone mill and bone graft delivery system (sometimes referred to as a “bone impactor system”). In one or more embodiments, the bone impactor system is configured to attach to a torque generation system. In at least one embodiment, the bone impactor system includes one or more bone mills and one or more cutting elements coupled to a tapered element. In one or more embodiments, the bone mill and the cutting element rotate via torque received via the torque generation system. In various embodiments, the bone mill and cutting element are configured to mill bone material into graft material. In at least one embodiment, the tapered element is configured to receive the graft material and deliver the graft material to a target site. In one or more embodiments, an end of the tapered element includes a tip having one or more openings to deliver the bone graft material to a target site. In various embodiments, a process for generating and delivering bone graft material to a desired surgical location includes connecting the impactor system to a torque generation system, positioning the tip of the tapered element at a target site, generating bone graft via processing bone material through the bone mill and the cutting element, and delivering bone graft material to the target site via the tapered element.

According to a first aspect, a bone impactor system, comprising: A) at least one hopper; and B) a sterile compartment configured to receive bone material from the at least one hopper, wherein the sterile compartment comprises: 1) a first end; 2) a second end opposite the first end; and 3) between the first end and the second end: i) at least one bone milling mechanism configured to: a) rotatably couple to a torque generation system; and b) upon activation of the torque generation system, mill the bone material; and ii) at least one rotary cutting mechanism rotatably coupled to the at least one bone milling mechanism and configured to, upon activation of the torque generation system, cut the bone material, wherein milling and cutting the bone material transforms the bone material into bone graft material; and 4) an opening at the second end wherein the bone graft material exits the sterile compartment.

According to a further aspect, the bone impactor system of the first aspect or any other aspect, wherein the sterile compartment further comprises, between the first end and a second end, at least size one filter configured to selectively filter the bone graft material based on a predetermined pore size.

According to a further aspect, the bone impactor system of the first aspect or any other aspect, wherein the at least one bone milling mechanism, the at least one rotary cutting mechanism, and the at least one size filter are centrally aligned along a longitudinal axis extending from the first end to the second end.

According to a further aspect, the bone impactor system of the first aspect or any other aspect, wherein the sterile compartment further comprises at least one tapered element between the first end and the second end, wherein the at least one tapered element defines the opening at the second end and is configured to: A) receive the bone graft material from the at least one size filter; and B) concentrate the bone graft material toward the opening at the second end.

According to a further aspect, the bone impactor system of the first aspect or any other aspect, wherein: A) the at least one size filter is rotatable coupled to the at least one bone milling mechanism or the at least one cutting mechanism; and B) the bone impactor system further comprises at least one secondary bone milling mechanism rotatably coupled to the at least one size filter and configured to mill the bone graft material upon activation of the torque generation system.

According to a further aspect, the bone impactor system of the first aspect or any other aspect, wherein the at least one secondary bone milling mechanism is further configured to, upon activation of the torque generation system, drive the bone graft material through the at least one tapered element and out the opening at the second end.

According to a further aspect, the bone impactor system of the first aspect or any other aspect, wherein the at least one secondary bone milling mechanism is integrally formed with the at least one tapered element.

According to a further aspect, the bone impactor system of the first aspect or any other aspect, wherein the at least one secondary bone milling mechanism comprises a plurality of helical structures that extend along at least a portion of the at least one tapered element.

According to a further aspect, the bone impactor system of the first aspect or any other aspect, wherein the at least one secondary bone milling mechanism is configured to rotate independently from the at least one tapered element.

According to a further aspect, the bone impactor system of the first aspect or any other aspect, further comprising a compression sleeve configured to attach to the sterile compartment, wherein a portion of the at least one tapered element extends through the compression sleeve.

According to a further aspect, the bone impactor system of the first aspect or any other aspect, further comprising: A) at least one array configured to be releasably secured to a target site, wherein: 1) the at least one array comprises a plurality of tracking markers; 2) the plurality of tracking markers are radiopaque; and 3) the sterile compartment comprises at least one additional tracking marker at the second end; B) an imaging system configured to generate image data corresponding to the at least one array and the at least one additional tracking marker; and C) a computing device connected to a display and configured to: 1) receive the image data from the imaging system; 2) generate a simulation of the target site, the at least one array, and at least the second end based on the image data; and 3) cause the display to render the simulation.

According to a further aspect, the bone impactor system of the first aspect or any other aspect, further comprising a coupling mechanism configured to couple the second opening to an implant.

According to a second aspect, a bone impactor system, comprising: A) at least one hopper configured to hold bone material; and B) a compartment, wherein the compartment comprises: 1) a first opening configured to connect the at least one hopper to the compartment; 2) at least one milling mechanism configured to: i) receive, via the first opening, the bone material from the at least one hopper; and ii) mill the bone material into a first set of bone graft material; 3) at least one rotary cutting mechanism configured to: i) receive the first set of bone graft material; and ii) cut the first set of bone graft material into a second set of bone graft material; 4) at least one size filter configured to: i) receive the second set of bone graft material; and ii) selectively filter the second set of bone graft material into a third set of bone graft material; and 5) at least one tapered element defining a second opening of the compartment and configured to: i) receive the third set of bone graft material; and ii) direct the third set of bone graft material out of the compartment via the second opening.

According to a further aspect, the bone impactor system of the second aspect or any other aspect, wherein the at least one rotary cutting mechanism is configured to receive the bone material from the at least one milling mechanism.

According to a further aspect, the bone impactor system of the second aspect or any other aspect, wherein the at least one bone milling mechanism comprises an auger.

According to a further aspect, the bone impactor system of the second aspect or any other aspect, wherein the at least one rotary cutting mechanism comprises at least two blades.

According to a further aspect, the bone impactor system of the second aspect or any other aspect, wherein the at least one hopper is a second sterile compartment.

According to a further aspect, the bone impactor system of the second aspect or any other aspect, further comprising: A) a computing device configured to: 1) track a position of the at least one tapered element relative to a target site based on image data from an imaging system; and 2) command a display to render a simulation of the position of the at least one tapered element relative to the target site; B) the imaging system configured to generate the image data via imaging at least one tracking marker and at least one additional tracking marker; C) the at least one tracking marker affixed to the at least one tapered element; and D) the at least one additional tracking marker releasably secured to the target site.

According to a further aspect, the bone impactor system of the second aspect or any other aspect, further comprising: A) tubing comprising a first end and a second end opposite the first end; and B) a coupling mechanism configured to: 1) couple the first end of the tubing to the second opening; and 2) couple the second end of the tubing to an implant, wherein the tubing is configured to: i) receive the third set of bone graft material via the second opening; and ii) deliver the third set of bone graft material into the implant.

According to a third aspect, a method for preparing and delivering a bone graft, comprising: A) inserting bone material into a bone impactor device; B) processing, via the bone impactor device, the bone material into bone graft material, wherein processing the bone material comprises: 1) milling the bone material via a first rotational element of the bone impactor device; 2) cutting the bone material via a second rotational element of the bone impactor device; and 3) filtering the bone material to isolate the bone graft material; and C) delivering, via the bone impactor device, the bone graft material to a target site.

According to a further aspect, the method of the third aspect or any other aspect, further comprising introducing, via the bone impactor device, at least one agent to the bone graft material prior to delivering the bone graft material.

According to a further aspect, the method of the third aspect or any other aspect, further comprising monitoring, via a sensor of the bone impactor device, a position of the bone impactor device during delivery of the bone graft material.

According to a further aspect, the method of the third aspect or any other aspect, further comprising selecting the bone impactor device from a kit based on the target site of the subject, wherein the kit comprises two or more bone impactor devices.

According to a further aspect, the method of the third aspect or any other aspect, further comprising connecting the bone impactor device to a torque generation system.

According to a further aspect, the method of the third aspect or any other aspect, further comprising: A) releasably securing at least one array to the target site, wherein the at least one array comprises radiopaque material; B) generating, via an imaging system, image data of the at least one array; C) receiving the image data at a computing device; D) tracking, via the computing device, the delivery of the bone graft material to the target site, wherein tracking the delivery comprises: 1) generating a simulation of the target site based on the image data; and 2) rendering the simulation on a display connected to the computing device.

According to a further aspect, the method of the third aspect or any other aspect, wherein the target site comprises an implant and the method further comprises: A) coupling the bone impactor device to the implant; and B) delivering the bone graft material into the implant via the bone impactor device.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 shows a perspective view of an exemplary impactor system, according to one embodiment of the present disclosure;

FIG. 2 shows an exploded view of an exemplary impactor system, according to one embodiment of the present disclosure;

FIG. 3 shows a perspective view of an exemplary impactor system, according to one embodiment of the present disclosure;

FIG. 4 shows a cross section of an exemplary impactor system, according to one embodiment of the present disclosure;

FIG. 5 show partial cross-sections of exemplary impactor systems, according to one embodiment of the present disclosure;

FIG. 6 also show partial cross-sections of exemplary impactor systems, according to one embodiment of the present disclosure;

FIG. 7 show shows an exemplary bone mill, according to one embodiment of the present disclosure;

FIG. 8 shows an exemplary bone graft delivery process, according to one embodiment of the present disclosure; and

FIG. 9 shows an exemplary graft generation process, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

As used herein, “bone material” includes, but is not limited to, autologous bone or allograft bone that has not been subjected to milling operations of the described bone impactor systems and processes.

As used herein, “bone graft material,” includes, but is not limited to, autologous bone or allograft bone that has been subjected to one or more milling operations of the described bone impactor systems and processes and, therefore, is suitable for delivery to a target site.

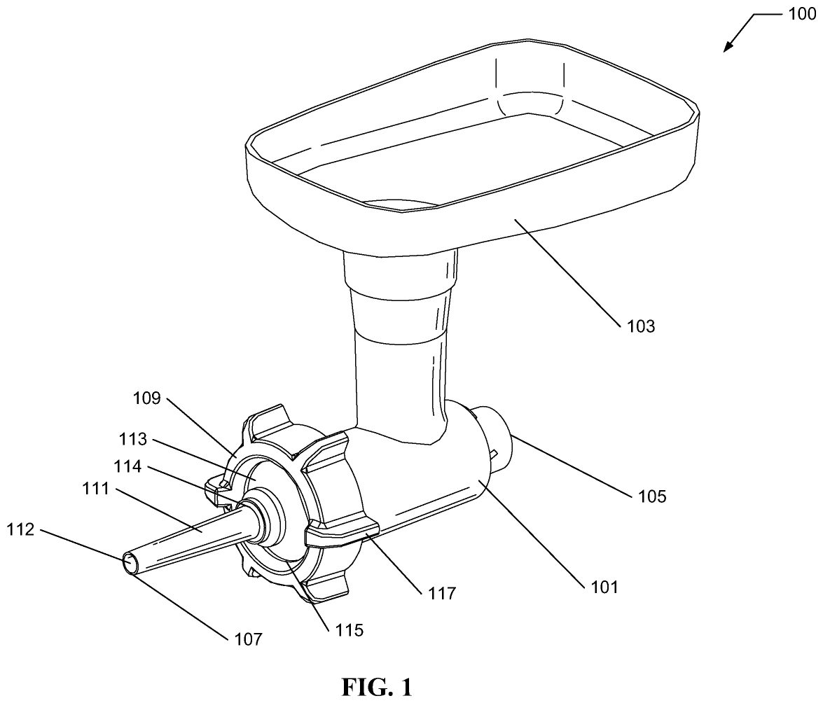

As an example, material loaded into a hopper of the described impactor system 100 (see FIG. 1) may be referred to as “bone material.” Continuing the example, following pulverization, cutting, and/or filtering of the bone material via the impactor system 100, the bone material is referred to as “bone graft material.”

In various embodiments, one or more gaskets, plates, tapering members, shafts, and connections therebetween, that are configured to rotatably couple one or more elements of the bone impactor system are collectively or individually referred to as “coupling members.”

### Overview

Aspects of the present disclosure generally relate to preparation and delivery of bone graft material.

The systems and methods described herein overcome the disadvantages of manually intensive conventional bone-mill systems and methods to deliver (implant) bone graft material via manual or array navigation to a desired location in a patient's body in a sterile environment. In one embodiment, the systems and methods described herein allow for the simultaneous generation and navigated delivery of bone graft material to a desired location in an open or minimally invasive procedure via a portable powered surgical bone mill and delivery system.

The present disclosure provides various embodiments of systems and processes for generating bone graft material and delivering bone graft material to a target site. In various embodiments, the system includes at least one array including tracking markers, the array releasably secured with a first anatomical feature. In one or more embodiments, the system includes at least one camera configured to track the array and transmit the images of the array to a computer system including a processor, wherein the computer system is configured to display a simulation of the anatomical feature on a display screen.

In at least one embodiment, provided herein is a process for guiding placement of the impactor system at a target site and for guiding the delivery of bone graft material from the impactor system to the target site. In various embodiments, the process includes providing at least one array releasably secured with the first vertebra of the spine. In one or more embodiments, the process includes providing a tracking system in communication with a navigation system including a computer system having a processor, wherein the tracking system captures images of the array and the spine and communicates the images to the computer system. In at least one embodiment, the process includes tracking the delivery of the impactor system and/or graft material into the surgical space with the tracking system. In various embodiments, the process includes communicating images of the delivered graft to the computer system.

### Exemplary Embodiments

Referring now to the figures, for the purposes of example and explanation of the fundamental processes and components of the disclosed systems and processes, reference is made to FIG. 1, which illustrates an exemplary, impactor system 100. As will be understood and appreciated, the impactor system 100 shown in FIG. 1 represents merely one approach or embodiment of the present system, and other aspects are used according to various embodiments of the present system.

FIG. 1 shows a perspective view of an exemplary impactor system 100. In at least one embodiment, the impactor system 100 is configured to receive bone material, mill the bone material into bone graft material, and deliver the bone graft material out of the system 100 to a target site. In one or more embodiments, the impactor system 100 extends between a first end 105 and a second end 107. In various embodiments, the impactor system 100 includes, but is not limited to, a body 101 and one or more hoppers 103. In at least one embodiment, the bone impactor system 100 (and other bone impactor systems shown and described herein) includes one or more materials including, but not limited to, metal, durable plastic, and composite materials. Non-limiting examples of metal include stainless steel, titanium, nickel, copper, and alloys derived therefrom. Non-limiting examples of plastic include acrylonitrile butadiene styrene (ABS), acetal copolymer, acetal homopolymer, polyethylene terphthalate polyester, polytetrafluoroethylene, ethylene-chlorotrifluoro-ethylene, polybutylene terephthalate-polyester, polyvinylidene fluoride, polyphenylene oxide, nylon, polycarbonate, one or more polyethylene, polypropylene homopolymer, polyphenylsulfone, polysulfone, polyethersulfone, and polyarylethersulfone. Non-limiting examples of composite materials include fiber-reinforced polymers, metal matrix composites, ceramic matrix composites, and thermoplastic composites. In at least one embodiment, the impactor system 100 includes one or more radiopaque materials including, but not limited to, barium sulfate, bismuth, tungsten, and iodine.

In one or more embodiments, the hopper 103 is configured to receive and hold bone material, such as, for example, autologous or allograft bone. In one example, a surgeon extracts autologous bone material from a hip, leg, rib, or other suitable portion of a subject. Continuing the example, the surgeon loads the autologous bone material into the impactor system 100 by inserting the autologous bone material into the hopper 103.

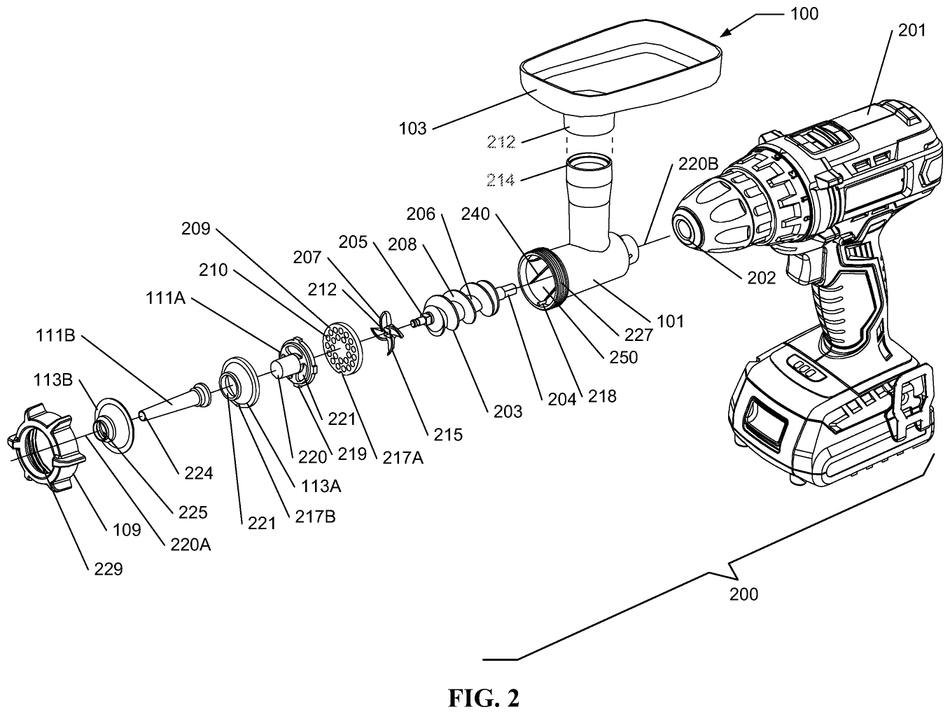

In at least one embodiment, the hopper 103 passes bone material into the body 101 via a connection between adjacent hopper and body chambers (not shown, see chambers 412, 414 shown in FIG. 2 and described herein). The impactor system 100 can include multiple hoppers 103. For example, the impactor system 100 includes a first hopper connected to the body 101 via a first aperture (not shown) and a second hopper connected to the body 101 via a second aperture (not shown) that is distinct from the first aperture. In another example, a first hopper 103 and a second hopper 103 connect to the body 101 via the same aperture.

In some embodiments, the position of the hopper 103 relative to the body 101 can be adjusted. For example, a connection between the hopper 103 and the body 101 includes a hinge or joint by which the hopper 103 can be moved from a first position to one or more secondary positions. In this example, the first position can refer to the hopper 103 being positioned directly over the body 101. In the same example, the secondary position can include the hopper 103 being positioned at an offset from the body 101 (e.g., 15 degrees offset, 30 degrees offset, or any suitable value). In at least one embodiment, the hopper 103 includes a cover (not shown). In various embodiments, the cover secures over the hopper 103 to prevent contamination thereof and maintain a sterile environment therein.

In at least one embodiment, bone material passes from the hopper 103 to the body 101 under force of gravity. For example, the hopper 103 is located superior to the body 101 such that gravitational forces direct bone material downward into the body 101. In some embodiments, the hopper 103 includes one or more mechanisms configured to direct bone material into the body 101. For example, the hopper 103 includes an arm configured to, upon activation, sweep across the hopper 103 and, thereby, contact and direct bone material from the hopper 103 into the body 101. In another example, the hopper 103 includes one or more vibratory elements that, upon activation, vibrate the hopper 103 and, thereby, cause bone material to shift and translate toward the body 101. In some embodiments, the hopper 103 includes markings and/or physical structures for facilitating accurate and precise apportionment of bone material.

In at least one embodiment, the impactor system 100 includes a tapered element 111 configured to deliver bone graft material from the impactor system 100 to a target site. In various embodiments, the body 101 is configured to receive the tapered element 111, or a portion thereof. In some embodiments, the tapered element 111 is referred to as a “tapering bore” or “access corridor.” In various embodiments, the tapered element 111 includes a cylindrical shape, a conical shape, a tubular shape, or any other suitable shape or shape combination. In some embodiments, the tapered element 111 tapers in external and/or internal diameter toward the second end 107. In one example, the tapered element 111 includes a cylindrical tube that decreases in diameter toward the second end 107.

In one or more embodiments, the tapered element 111 includes a chamber 112. In various embodiments, the chamber 112 is a void extending along a length of the tapered element 111. In at least one embodiment, the chamber 112 is configured to receive bone graft material (e.g., or an intermediary material, such as partially milled bone) from one or more bone milling, bone cutting, and/or bone filtering elements within the body 101. In some embodiments, the chamber 112 includes one or more bone milling, bone cutting, and/or bone filtering elements. For example, the body 101 includes a first bone mill towards the first end 105 and a second bone mill toward the second end 107, the second bone mill being housed within (or including) the chamber 112 of the tapered element 111.

In at least one embodiment, the impactor system 100 includes one or more gaskets 113 configured to preserve the sterile environment within the body 101. In one or more embodiments, the gasket 113 includes a shaped piece or ring of rubber or other material that seals a connection between the body 101 and the tapered element 111. In various embodiments, the gasket 113 includes an opening 114. In at least one embodiment, the tapered element 111, or a portion thereof, passes through the opening 114.

In one or more embodiments, the impactor system 100 includes an attachment element 109 configured to secure to the body 101. In some embodiments, the attachment element 109 is referred to as a “compression sleeve.” In at least one embodiment, the attachment element 109 is configured to secure to the body 101 via any suitable means including, but not limited to, threads, luer lock or bayonet fitting, snap fitting, press fitting, adhesives, or magnets. In one or more embodiments, the attachment element 109 is configured to slide over the tapered element 111 and the gasket 113. In at least one embodiment, the attachment element 109 is configured to secure to the body 101 and, thereby, maintain a position of the tapered element 111 and the gasket 113. In various embodiments, the attachment element 109 includes an opening 115. In at least one embodiment, the opening 115 is configured to receive the tapered element 111 and the gasket 113, or portions thereof. In one or more embodiments, the attachment element 109 includes one or more ridges 117 for providing gripping means to a user of the impactor system 100.

FIG. 2 shows an exploded view of the impactor system 100. In various embodiments, the impactor system 100 includes, but is not limited to, the hopper 103, the body 101, the attachment element 109, one or more tapered elements 111A,B, one or more gaskets 113A,B, one or more bone mills 203, one or more cutting elements 207, and one or more size filters 209. In at least one embodiment, the impactor system 100 includes a longitudinal axis 220A, B. In various embodiments, one or more of the body 101, attachment element 109, tapered elements 111A, 111B, gaskets 113A, 113B, bone mill 203, cutting element 207, and size filter 209 are centrally aligned along the longitudinal axis 220A. In one or more embodiments, one or more of the bone mill 203, cutting element 207, size filter 209, tapered elements 111A, 111B, and gaskets 113A, 113B are rotatably coupled.

In one or more embodiments, the body 101 includes one or more shapes including, but not limited to, prisms, cylinders, and other solids of revolution. For example, the body 101 includes a cylindrical, tubular shape. In various embodiments, the body 101 is configured to receive and house one or more of the bone mill 203, cutting element 205, size filter 207, tapered elements 111A, 111B, and gaskets 113A, 113B. In one or more embodiments, the body 101 includes a void 250 configured to receive one or more of the bone mill 203, cutting element 205, size filter 207, tapered elements 111A, 111B, and gaskets 113A, 113B.

In at least one embodiment, the bone mill 203 is configured to mill bone material that has been received into the body 101 from the hopper 103. In various embodiments, the bone mill 203 rotates to mill the bone material. In some embodiments, the body 101 includes one or more mill structures 240 that extend from the body 101 into the void 250. In at least one embodiment, the bone mill 203 is configured to rotate bone material against the mill structure 240 to cut, crush, or pulverize bone. In various embodiments, the mill structures 240 include a helical pattern or other pattern based on the orientation and arrangement of the bone mill 203, or elements thereof. In some embodiments, a bone mill (e.g., or components thereof, such as a shaft and one more blades that extend from the shaft) is referred to as an “auger.”

In one or more embodiments, the bone mill 203 is configured to receive torque via a torque generation system 201. In at least one embodiment, the bone mill 203 includes a first end 204 and a second end 205 opposite the first end 204. In one or more embodiments, the bone mill 203 includes a shaft 206 and one or more blades 208 between the first end 204 and the second end 205. In various embodiments, the blades 208 are integrally formed with or attached to the shaft 206. In one at least one embodiment, the first end 204 defines the end 105 of the impactor system 100.

In one or more embodiments, the impactor system 100 is configured to attach to a torque generation system 201. In various embodiments, the torque generation system 201 is configured to generate constant rotational force at one or more force levels. In one or more embodiments, the torque generation system 201 includes a motor or other means for generating a rotational force. The torque generation system 201 can operate via electrical power, pneumatic power, manual power, or combinations thereof. In one example, the torque generation system 201 includes a motorized drill. In at least one embodiment, the bone mill 203 is configured to rotatably couple to the torque generation system 201. In one example, the bone mill 203 is configured to rotatably couple to the torque generation system 201 at the first end 204 and rotatably couple to the cutting element 209 at the second end 205. In various embodiments, the first end 204 is configured connect the torque generation system 201 and transfer torque from the torque generation system 201 to one or more bone milling, bone cutting, and/or bone filtering elements of the impactor system 100. In one or more embodiments, the first end 204 is configured for receipt into an adjustable opening 202 of the torque generation system 201. The adjustable opening 202 can change diameter via any suitable means (e.g., threads, struts, etc.). In at least one embodiment, the first end 204 includes a shank shape configured for receipt into the adjustable opening 202. For example, the first end 204 includes a hexagonal shape and the opening 202 includes surfaces configured to contact the hexagonal shape upon adjustment of the diameter of the opening 202. Non-limiting examples of shank shapes include brace shank, straight shank, hex shank, slotted drive shaft (SDS) shank, morse taper shank, square shank, and threaded shank. In some embodiments, the impactor system 100 includes an additional shaft including a first end configured to attach to the torque generation system 201 and a second end configured to attach to the first end 204 (e.g., thereby enabling transfer of torque from the torque generation system 201 to the bone mill 203.).

In at least one embodiment, the second end 205 of the bone mill 203 is configured to connect to one or more of the cutting element 207, size filter 209, tapered element 111A, and tapered element 111B. In at least one embodiment, the cutting element 207 includes a void 212 configured to receive the second end 205. In various embodiments, the cutting element 207 is configured to receive torque from the bone mill 203 via receipt of the second end 205 into the void 212. The void 212 and second end 205 can each include a similar shape or combination of shapes for enabling receipt of the second end 205 into the void 212 and transfer of torque from the bone mill 203 to the cutting element 207. In one example, the second end 205 and void 212 each include a square shape (e.g., the square of the void 212 being slightly larger to accommodate the square shape of the second end 205).

In various embodiments, the cutting element 205 includes one or more blades 215. The cutting element 205 can include 1, 2, 3, 4, or any suitable number of blades 215. The blades 215 can demonstrate identical or varying dimensions. For example, a first blade 215 can include a length that exceeds a length of a second blade 215. In another example, a first blade 215 includes a first pitch that is less than a pitch of a second blade 215. In one or more embodiments, the cutting element 205 rotates to cut bone material into bone graft material. In one example, the cutting element 205 receives milled bone material from the bone mill 203 and cuts the milled bone material into bone graft materials (e.g., or an intermediary bone material).

In at least one embodiment, the size filter 209 is configured to receive and filter bone material, intermediary bone material, or bone graft material from the cutting element 205 and/or the bone mill 203. In various embodiments, the size filter 209 includes pores 210 that extend through the size filter 209. In one or more embodiments, the size filter 209 is configured to a) allow bone material beneath a predetermined size to pass through the size filter 109, and b) prevent bone material at or above the predetermined size from passing through the size filter 209 (e.g., until such material is further cut and/or milled to a size than the predetermined size). In one or more embodiments, the pores 210 are arranged into a random or predetermined pattern about the size filter 209. In one example, the pores 210 are arranged into concentric radial patterns. In another example, the pores 210 are arranged into a spiral pattern. In at least one embodiment, each pore 210 includes a diameter that measures at least 0.5 mm, 0.5-20.0 mm, 0.5-1.0 mm, 1.0-2.0 mm, 2.0 mm, 2.0-4.0 mm, 4.0-6.0 mm, 5.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In some embodiments, the pores 210 include variable diameters. For example, the pores 210 include a first subset of a first diameter and a second subset of a second diameter that exceeds the first diameter.

In various embodiments, the size filter 209 and the body 101 include mated structures for maintaining a position or orientation of the size filter 209 within the body 101. In at least one embodiment, the size filter 209 includes a channel 217A. In one or more embodiments, the body 101 includes a ridge 218. In at least one embodiment, the ridge 218 includes a shape that is substantially similar to a footprint of the channel 217A, thereby allowing the ridge 218 to fit into the channel 217A. In some embodiments, the mated structures are reversed such that the size filter 209 includes a ridge and the body 101 includes a channel. In various embodiments, the size filter 209 includes a plurality of channels 217A and the body 101 includes a plurality of ridges 218 configured to mate with the plurality of channels 217A.

In one or more embodiments, the tapered element 111A is configured to receive bone graft material from one or more of the size filter 209, cutting element 207, and bone mill 203. In at least one embodiment, the tapered element 111A is configured to concentrate the bone graft material toward the tapered element 111B and/or toward the second end 107 of the impactor system 100 shown in FIG. 1. In various embodiments, the tapered element 111A includes a plate 219 and a shaft 220. According to one embodiment, the plate 219 is integrally formed with the shaft 220 or is attached to the shaft 220 via any suitable means (e.g., adhesives, fasteners, press fitting, snap fitting, etc.). In at least one embodiment, the plate 219 includes one or more voids 221 configured to pass bone graft material through the tapered element 111A. In one example, the plate 219 includes three voids arranged radially around the plate 219. The void 221 can include any suitable shape, such as circles, rounded triangles, or fabiforms. In various embodiments, the shaft 220 is configured to contact the gasket 113A and form a spatial offset between the plate 219 and the gasket 113A and/or the tapered element 111B. In at least one embodiment, the offset between the plate 219 and the gasket 113A and/or tapered element 111B includes dimensions sufficient for allowing passage of bone graft material from the tapered element 111A into the tapered element 111B.

In at least one embodiment, the gaskets 113A, 113B include any suitable shape, such as, for example, a cylinder or disc. In various embodiments, the gasket 113A includes an opening 221. In one or more embodiments, via the opening 221, the gasket 113A is configured to slide over the tapered element 111B and/or the tapered element 111A (e.g., or slide over portions thereof). In various embodiments, the gasket 113A is configured to maintain a sterile environment between components of the impactor system 100 and an external environment. In at least one embodiment, a diameter of the opening 221 is substantially similar to a diameter of the tapered elements 111A, 111B such that the gasket 113A forms a seal between each tapered element 111A, 111B. In at least one embodiment, the gasket 113A includes one or more channels 217B. In various embodiments, the channel 217B is configured to mate with the ridge 218 of the body 101 and, thereby, maintain a position or orientation of the tapered element 111A within the body 101.

In at least one embodiment, the tapered element 111B is configured to receive bone graft material from one or more of the tapered element 111A, the size filter 209, cutting element 207, and bone mill 203. In various embodiments, the tapered element 111A is configured to concentrate the bone graft material toward the second end 107 of the impactor system 100 shown in FIG. 1. In one or more embodiments, the tapered element 111B includes a tip 224. In various embodiments, the impactor system 100 delivers bone graft material to a target site via the tip 224. In some embodiments, the tip 224 includes an angle or taper. According to one embodiment, the tip 224 is curved to enable delivery of bone graft material to improve precise and accurate delivery of bone graft material to a target site and/or to enable delivery of bone graft material to otherwise inaccessible target sites. In at least one embodiment, the tip 224 includes one or more radiopaque materials that enable the tip 224 to be observed via one or more imaging systems and/or techniques (e.g., X-ray, magnetic resonance, computed tomography, ultrasound, etc.). For example, the tip 224 includes barium sulfate. In one or more embodiments, the tip 224 is blunt, rounded, or chamfered.

In some embodiments, the tip 224 includes or is connected to tubing that is configured for passing bone graft material out of the impactor system 100 and into a target site. Non-limiting examples of the tubing include catheters and lumens. The tubing can be flexible or rigid. In at least one embodiment, the tubing includes one or more permanent bends or curves. In various embodiments, the tubing may be used to provide precise and accurate delivery of bone graft material (e.g., to a target site and/or into an implant at a target site). For example, during a minimally invasive surgery, a surgeon inserts the tubing into a subject via an incision and directed to a target site. Continuing the example, the surgeon generates bone graft material via the impactor system 100 and delivers the bone graft material from the impactor system 100 to the target site via the tubing. In at least one embodiment, the impactor system 100 includes a coupling member configured to secure tubing to the tip 224 and/or other elements of the impactor system 100. Non-limiting examples of coupling members include bayonet fittings, snap- and/or press-fit connectors, and winch straps. In various embodiments, the tubing may be coupled to an implant, thereby allowing the impactor system 100 to deliver bone graft material into one or more portions of the implant. In one example, a first end of tubing is coupled to the tip 224 and a second end of the tubing is coupled to an interbody implant positioned within a vertebral disc space. Continuing the example, the impactor system 100 receives bone material, transforms the bone material into bone graft material, and delivers the bone graft material directly into the interbody implant via the tubing. In at least one embodiment, the delivery of the bone graft material via tubing advantageously supports minimally invasive surgical procedures.

In one or more embodiments, the impactor system includes at least one end that is configured to connect to implantable hardware, such as inter-disc devices, expandable cages, screws, and joint fusion devices. In various embodiments, the impactor system connects to the implantable hardware via a coupling mechanism. In at least one embodiment, the coupling between the impactor system and implantable hardware includes a mechanism for providing targeted disconnection of the impactor system. The mechanism can include any suitable means for providing a releasable connection, such as, for example, snap fittings or bayonet or luer-lock fittings.

In one or more embodiments, the gasket 113B includes an opening 225. In at least one embodiment, via the opening 225, the gasket 113B is configured to slide over the tapered element 111A (e.g., or slide over portions thereof). In various embodiments, the gasket 113A is configured to maintain a sterile environment between components of the impactor system 100 and an external environment. In one or more embodiments, the gasket 113B is configured to maintain a sterile environment within the body 101.

In at least one embodiment, the attachment element 109 is configured to slide over the gaskets 113A, 113B and tapered elements 111A and secure to the body 101. In one or more embodiments, the body 101 includes threads 227 and the attachment element 109 includes threads 229 configured to mate with the threads 227.

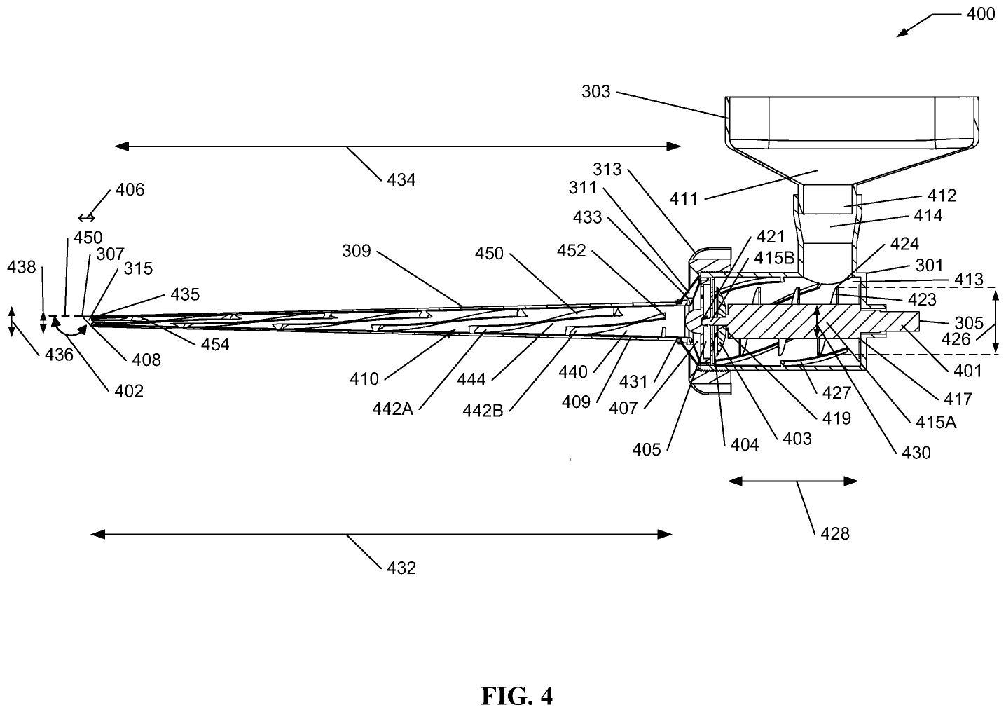

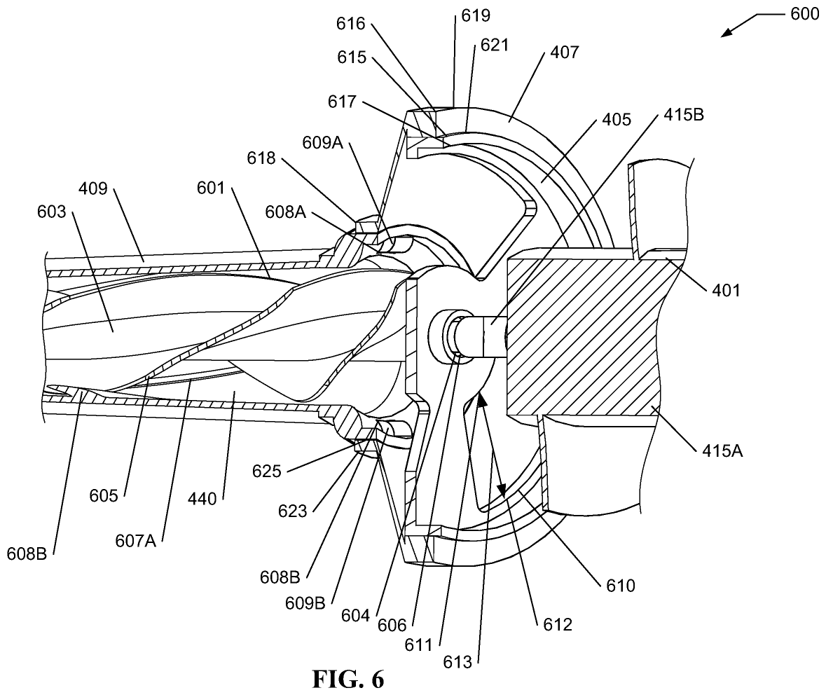

In various embodiments, the tapered element 111B and/or the tapered element 111A include one or more bone mills (not shown). For example, the tapered element 111A includes a bone mill (see, for example, bone mill 410 and bone mill 601 shown in FIGS. 4 and 6, respectively, described herein).

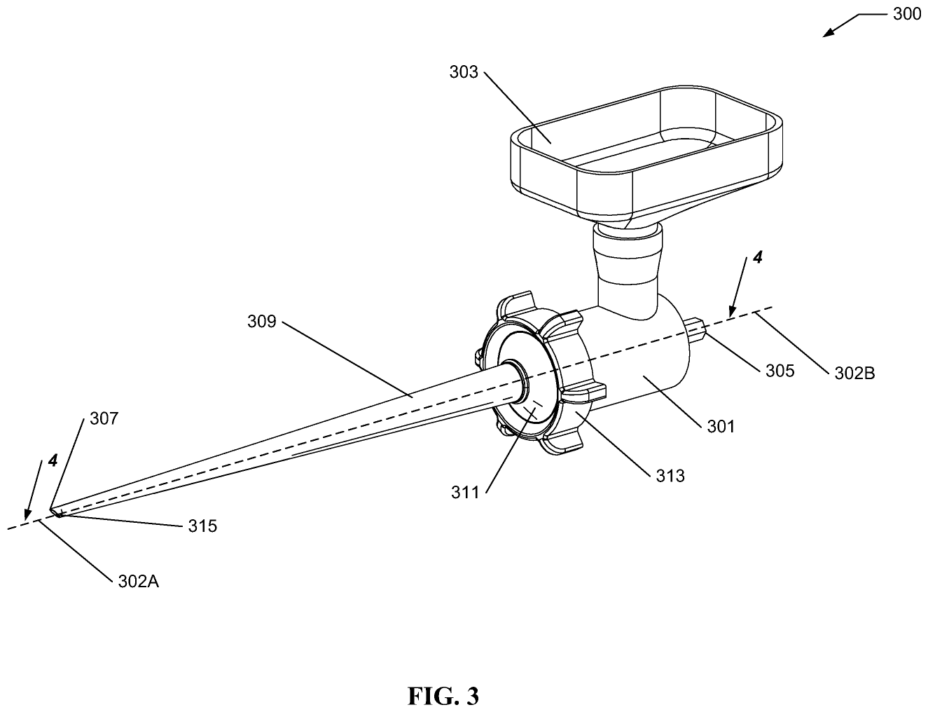

FIG. 3 shows a perspective view of an exemplary impactor system 300. In at least one embodiment, the impactor system 300 is similar to the impactor system 100 shown in FIGS. 1-2. In various embodiments, one or more elements of the impactor system 300 demonstrate one or more properties and perform one or more functions of similarly named elements of the impactor system 100. In one or more embodiments, the impactor system 300 includes, but is not limited to, a body 301, one more hoppers 303, an exterior tapered element 309 (also referred to as a “sheath”), one or more gaskets 311, and an attachment element 313.

In various embodiments, the impactor system 300 extends between a first end 305 and a second end 307. In one or more embodiments, the impactor system 300 is configured to connect to a torque generation system (e.g., the torque generation 201 shown in FIG. 2) at the first end 305. In at least one embodiment, one or more of the body 301, exterior tapered element 309, gasket 311, and attachment element 313 are centrally oriented about a longitudinal axis defined by a reference line 302A, 302B. In various embodiments, the reference line 302A, 302B indicates cross sections 400, 500, and 600 of the impactor system 300 shown in FIGS. 4, 5, and 6, respectively. In one or more embodiments, as shown in FIG. 4, the impactor system 300 includes various elements configured for milling, cutting, and filtering bone into bone graft material and various elements for concentrating and delivering bone graft material to a target site.

FIG. 4 shows an exemplary cross section 400 of the impactor system 300 (see FIG. 3). In various embodiments, the impactor system 300 includes, but is not limited to, the body 301, hopper 303, exterior tapered element 309, gasket 311, attachment element 313, one or more bone mills 401, one or more cutting elements 403, one or more size filters 404, a plate 405, a gasket 407, an interior tapered element 409, and one or more bone mills 410. In various embodiments, one or more of the hopper 303, exterior tapered element 309, gasket 311, attachment element 313, bone mill 401, cutting element 403, size filter 404, gasket 407, interior tapered element 409, and bone mill 410 are rotatably coupled. In one or more embodiments, the bone mill 401 includes a first shaft 415A and a second shaft 415B.

In one or more embodiments, the plate 405 is rotatably coupled to the bone mill 410 and the gasket 407. In one or more embodiments, the plate 405 is configured to receive the second shaft 415B of the bone mill 401. In various embodiments, the plate 405 is configured to secure within the gasket 407. In at least one embodiment, the interior tapered element 409 is configured to attach to the gasket 407, thereby rotationally coupling the interior tapered element 409 to the bone mill 401. In some embodiments, the interior tapered element 409 is configured to attach to the plate 405. In various embodiments, connections between the gasket 407 and the interior tapered element 409, between the gasket 407 and the plate 405, and between the plate 405 and the second shaft 415B allow the interior tapered element 409 to rotate in response to rotation of the bone mill 410. In some embodiments, the bone mill 401 is rotatably coupled to a bone mill 601 (not shown, see cross section 600 shown in FIG. 6).

In various embodiments, the hopper 303 includes a chamber 411 configured to receive bone material. In one or more embodiments, the body 401 includes a chamber 413 configured to receive bone material from the chamber 411. In one or more embodiments, the hopper 303 includes an opening 412 and the body 301 includes an opening 414, the openings 412, 414 being configured to permit passage of bone material from the chamber 411 into the chamber 413.

In one or more embodiments, the exterior tapered element 309 sheathes one or more of the interior tapered element 409 (e.g., and bone mill 410 located therein) and gasket 407. In at least one embodiment, the exterior tapered element 309 is secured against rotation. For example, the exterior tapered element 309 is secured against rotation such that the interior tapered element 409 may rotate freely within the exterior tapered element 309 while the exterior tapered element 309 remains rotationally static. In one or more embodiments, the tip 315 of the exterior tapered element 309 includes an opening configured to pass bone graft material from the interior tapered element 409, out of the impactor system 300, and to a target site. In various embodiments, the tip 315 includes a taper angle 402 as measured from a horizontal plane defined by a reference line 450. In one or more embodiments, the taper angle 402 measures 0 degrees, 0-5 degrees, at least 5.0 degrees, 5.0-355.0 degrees, 5.0-15.0 degrees, 15.0-30.0 degrees, 30.0-45.0 degrees, 40.3 degrees, 48.0 degrees, 45.0-60.0 degrees, 60.0-75.0 degrees,75.0-90.0 degrees, 90.0-105.0 degrees, 105.0-120.0 degrees, 120.0-135.0 degrees, 135.0-150.0 degrees, 150.0-165.0 degrees, 165.0-180.0 degrees, 180.0-195.0 degrees, 195.0-210.0 degrees, 210.0-225.0 degrees, 225.0-240.0 degrees, 240.0-255.0 degrees, 255.0-270.0 degrees, 270.0-285.0 degrees, 285.0-300.0 degrees, 300.0-315.0 degrees, 315-330.0 degrees, 330-345.0 degrees, 345-355.0 degrees, less than 355.0 degrees, or 355.0-360.0 degrees. In one or more embodiments, the tip 315 includes a length 406 between a first chamber second end 408 and the second end 307. In at least one embodiment, the length 406 measures at least 2.0 mm, 2.0-30.0 mm, 2.0-4.0 mm, 4.0-6.0 mm, 5.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, 20.0-22.0 mm, 22.0-24.0 mm, 24.0-26.0 mm, 26.0-28.0 mm, 28.0-30.0 mm, or less than 30.0 mm.

In at least one embodiment, the exterior tapered element 309 includes a length 432 between the end 307 and an end 431. In various embodiments, the length 432 measures at least 100.0 mm, 100.0-500.0 mm, 100.0-150.0 mm, 150.0-200.0 mm, 200.0-250.0 mm, 250.0-300.0 mm, 294.0 mm, 300.0-350.0 mm, 313.0 mm, 350.0-400.0 mm, 400.0-450.0 mm, 450.0-500.0 mm, or less than 500.0 mm. In at least one embodiment, the interior tapered element 409 includes a length 434 between a first chamber first end 433 and a second end 435. In some embodiments, the length 434 is less than or equal to the length 432.

In one or more embodiments, the exterior tapered element 309 includes a first diameter 436 at the end 431 and a second diameter 438 at the chamber second end 408. In various embodiments, the first diameter 436 is less than, greater than, or equal to the second diameter 438. For example, the exterior tapered element 309 tapers from the first diameter 436 to the second diameter 438. In one or more embodiments, the first diameter 436 measures at least 5.0 mm, 5.0-50.0 mm, 5.0-10.0 mm, 10.0-15.0 mm, 16.0 mm, 15.0-20.0 mm, 20.0-25.0 mm, 25.0-30.0 mm, 30.0-35.0 mm, 35.0-40.0 mm, 40.0-45.0 mm, 45.0-50.0 mm, or less than 50.0 mm. In at least one embodiment, the second diameter 438 measures at least 1.0 mm, 1.0-50.0 mm, 1.0-5.0 mm, 3.6 mm, 5.0-10.0 mm, 10.0-15.0 mm, 16.0 mm, 15.0-20.0 mm, 20.0-25.0 mm, 25.0-30.0 mm, 30.0-35.0 mm, 35.0-40.0 mm, 40.0-45.0 mm, 45.0-50.0 mm. In various embodiments, the interior tapered element 409 includes one or more diameters less than or equal to the first diameter 436 and the second diameter 438. In one example, the interior tapered element 409 includes a first diameter between 15.0-20.0 mm toward the chamber first end 433 and a second diameter between 1.0-5.0 mm toward the chamber second end 408.

In at least one embodiment, the exterior tapered element 309 tapers from the first diameter 436 to the second diameter 348 at particular rate and a particular angle. In various embodiments, the particular rate is at least 0.1 mm/mm (e.g., X mm of taper per 1.0 mm of length), 0.1-4.0 mm/mm, 0.1-0.5 mm/mm, 0.5-1.0 mm/mm, 1.0 mm/mm, 1.0-1.5 mm/mm, 1.5-2.0 mm/mm. 2.0-2.5 mm/mm, 2.5-3.0 mm/mm, 3.0-3.5 mm/mm. 3.5-4.0 mm/mm, or less than 4.0 mm/mm. In various embodiments the particular angle (e.g., as measured from a plane defined by reference line 402) measures 0.0 degrees, at least 1.0 degrees, 0.1-30.0 degrees, 0.1-2.0 degrees, 2.0-4.0 degrees, 2.3 degrees, 4.0-6.0 degrees, 6.0-8.0 degrees, 8.0-10.0 degrees, 10.0-12.0 degrees, 12.0-14.0 degrees, 14.0-16.0 degrees, 16.0-18.0 degrees, 18.0-20.0 degrees, 20.0-22.0 degrees, 22.0-24.0 degrees, 24.0-26.0 degrees, 26.0-28.0 degrees, 28.0-30.0 degrees, or less than 30.0 degrees. In one or more embodiments, the interior tapered element 409 includes a taper rate and/or a taper angle similar to the particular rate and particular angle of the exterior tapered element 309.

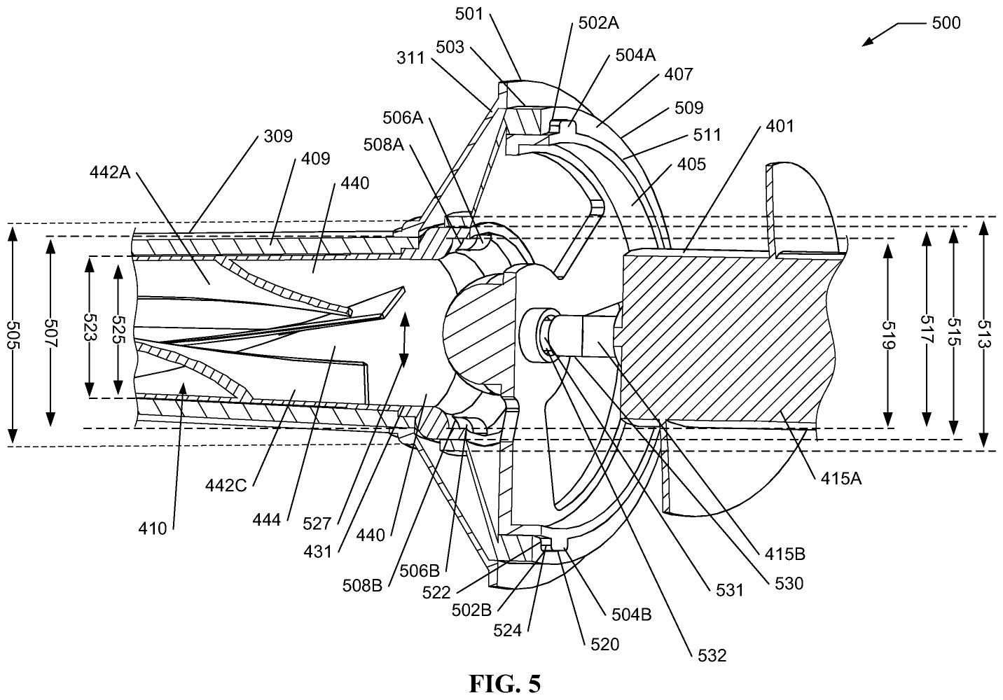

In at least one embodiment, the interior tapered element 409 includes a chamber 440 that extends from the chamber first end 433 to the chamber second end 408. In various embodiments, the chamber 440 is hollow such that bone graft material from the chamber 413 of the body 101 may pass into the interior tapered element 409. In one or more embodiments, the interior tapered element 409 includes a bone mill 410 within the chamber 440. In one or more embodiments, the bone mill 410 includes a plurality of blades 442A, 442B. In various embodiments, the blades 442A, 442B are integrally formed with or attached to the interior tapered element 409. In at least one embodiment, the blades 442A, 442B extend into the chamber 440. In various embodiments, each blade 442A, 442B includes a full or partial spiral shape, such as, for example a full or partial helix. In one or more embodiments, the bone mill 410 is configured to mill bone material or bone graft material via rotation of the interior tapered element 409. In at least one embodiment, the bone mill 410 is configured to advance bone graft material from the body 101 to the tip 315 via rotation of the interior tapered element 409. For example, upon rotation of the interior tapered element 409, the blades 442A, 442B simultaneously mill and advance bone graft material toward the end 307. In at least one embodiment, the interior tapered element 409 includes a void 444 that extends approximately from the end 431 to the chamber second end 408. In one or more embodiments, the void 444 is a central region of the chamber 440 into which the blades 442A, 442B do not extend. Exemplary dimensions of the void 444 are shown in and described with reference to FIG. 5.

In one or more embodiments, the blades 442A, 442B include a pitch 450 that measures at least 100.0 mm, 100.0-500.0 mm, 100.0-150.0 mm, 150.0-200.0 mm, 200.0-250.0 mm, 250.0-300.0 mm, 280.0 mm, 300.0-350.0 mm, 313.0 mm, 350.0-400.0 mm, 400.0-450.0 mm, 450.0-500.0 mm, or less than 500.0 mm. In some embodiments, the blades 442A, 442A includes a tapering pitch 450 from the end 431 to the second end 307, or vice versa. In one or more embodiments, the blades 442A, 442B taper in height from the end 431 to the second end 307, or vice versa. In at least one embodiment, the blades 442A, 442B include a first height 452 toward the end 431 and a second height 454 toward the second end 307. In various embodiments, the first height 452 measures at least 0.1 mm, 0.1-20.0 mm, 0.1-1.0 mm, 1.0-2.0 mm, 2.0-4.0 mm, 4.0-6.0 mm, 4.95 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In one or more embodiments, the second height 454 measures at least 0.1 mm, 0.1-20.0 mm, 0.1-1.0 mm, 1.0-2.0 mm, 1.25 mm, 2.0-4.0 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm.

In various embodiments, the bone mill 401 includes a shaft 415A and a shaft 415B integrally formed with or rotationally coupled to the shaft 415A. In one or more embodiments, the shaft 415A extends between a first end 417 and a second end 419. In various embodiments, the shaft 415B extends between the second end 419 and a third end 421. In one or more embodiments, the shafts 415A, 415B include generally cylindrical shapes. In various embodiments, the shaft 415 includes one or more shapes configured to rotationally couple the bone mill 401 to one or more of the cutting element 403, size filter 404, gasket 407, and gasket 311. In one example, the shaft 415 rotationally couples to the cutting element 403 and the gasket 407.

In one or more embodiments, the shaft 415A includes one or more blades 423 between the first end and second end. The shaft 415A can include 1, 2, 3, 4, or any suitable number of blades 423. The blade 423 can be integrally formed with or attached to the shaft 415A. In at least one embodiment, the blade 423 includes one or more sharpened threads. In various embodiments, the blade includes a constant or varying pitch 424. For example, the pitch 424 increases from the first end 417 to the second end 419, or vice versa. In various embodiments, the pitch 424 measures at least 5.0 mm, 5.0-50.0 mm, 5.0-10.0 mm, 10.0-15.0 mm, 15.0-20.0 mm, 20.0 mm, 20.0-25.0 mm, 25.0-30.0 mm, 30.0-35.0 mm, 35.0-40.0 mm, 40.0-45.0 mm, 45.0-50.0 mm, or less than about 50.0 mm. In one or more embodiments, the blade includes a constant or varying diameter 426. In at least one embodiment, the diameter 426 measures at least 5.0 mm, 5.0-50.0 mm, 5.0-10.0 mm, 10.0-15.0 mm, 15.0-20.0 mm, 20.0-25.0 mm, 22.6 mm\, 25.0-30.0 mm, 30.0-35.0 mm, 35.0-40.0 mm, 40.0-45.0 mm, 45.0-50.0 mm, or less than 50.0 mm.

In some embodiments, the body 101 includes one or more mill structures 427 that extend into the chamber 413 and are configured to interface with the bone mill 401 (e.g., in particular, with the one or more blades 423 thereof). In at least one embodiment, the mill structures 427 include a helical or threaded shape. In one or more embodiments, the mill structures 427 are configured for crushing bone material between the mill structures 427 and the blade 423.

In various embodiments, the shaft 415A includes a length 428 between the first end 417 and the second end 419. In one or more embodiments, the length 428 measures at least 100.0 mm, 100.0-400.0 mm, 100.0-125.0 mm, 125.0-150.0 mm, 150.0-175.0 mm, 175.0-200.0 mm, 200.0-225.0 mm, 225.0-250.0 mm, 250.0-275.0 mm, 274.0 mm, 275.0-300.0 mm, 300.0-325.0 mm, 325.0-350.0 mm, 350.0-375.0 mm, 375.0-400.0 mm, or less than 400.0 mm. In one or more embodiments, the shaft 415A includes a diameter 430 that measures at least 5.0 mm, 5.0-50.0 mm, 5.0-10.0 mm, 10.0-15.0 mm, 15.0 mm, 15.0-20.0 mm, 20.0-25.0 mm, 25.0-30.0 mm, 30.0-35.0 mm, 35.0-40.0 mm, 40.0-45.0 mm, 45.0-50.0 mm, or less than 50.0 mm.

FIG. 5 shows a partial cross-section 500 of the impactor system 300 (see FIG. 3). In one or more embodiments, the gasket 407 includes recesses 502A, 502B. In various embodiments, the plate 405 includes tabs 504A, 504B. In various embodiments, the recesses 502A, 502B are configured to receive the tabs 504A, 504B to rotatably couple the gasket 407 to the plate 405. In various embodiments, the plate 405 includes any suitable number of tabs (e.g., 1, 3, 5, etc.) and the gasket 407 includes an equal number of recesses configured to receive the tabs. In one or more embodiments, the recesses 502A, 502B include a length 520 that measures at least 1.0 mm, 1.0-20.0 mm, 1.0-2.0 mm, 2.0-4.0 mm, 3.5 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In various embodiments, the recesses 502A, 502B include a height 522 that measures at least 0.1 mm, 0.1-20.0 mm, 0.1-1.0 mm, 1.0-2.0 mm, 1.3 mm, 2.0-4.0 mm, 3.5 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In one or more embodiments, the recesses 502A, 502B and tabs 504A, 504B include a depth 524 that measures at least 1.0 mm, 1.0-20.0 mm, 1.0-2.0 mm, 2.0-4.0 mm, 4.0 mm, 4.0-6.0 mm, 5.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In some embodiments, the recesses 502A, 502B include a depth that is less than, greater than, or equal to a depth of the tabs 504A, 504B. For example the recesses 502A, 502B include a depth of 4.0 mm and the tabs 504A, 504B include a depth of 5.0 mm.

In various embodiments, the gasket 407 rotatably couples to the interior tapered element 409 via one or more snap fittings. In at least one embodiment, each snap fitting includes a prong and a slot configured to receive the prong. In one or more embodiments, the gasket 407 includes prongs 506A, 506B. In various embodiments, the interior tapered element 409 includes slots 508A, 508B configured to receive the prongs 506A, 506B. According to one embodiment, each slot 506A, 506B is a void and each void includes a shape similar to a footprint of the prongs 506A, 506B. The gasket 407 can include any suitable number of prongs (e.g., 1, 3, 5, etc.) and the interior tapered element 409 includes an equal number of slots (e.g., 1, 3, 5, etc.). In some embodiments, the interior tapered element 409 includes prongs and the gasket 407 includes slots configured to receive the prongs.

In one or more embodiments, the gasket 311 includes a first outer diameter 501 and a first inner diameter 503. In at least one embodiment, the first outer diameter 501 measures at least 5.0 mm, 5.0-100.0 mm, 5.0-10.0 mm, 10.0-20.0 mm, 20.0-30.0 mm, 30.0-40.0 mm, 40.0-50.0 mm, 50.0 mm, 50.0-60.0 mm, 60.0-70.0 mm, 70.0-80.0 mm, 80.0-90.0 mm, 90.0-100.0 mm, or less than 100.0 mm. In various embodiments, the first inner diameter 503 measures at least 5.0 mm, 5.0-100.0 mm, 5.0-10.0 mm, 10.0-20.0 mm, 20.0-30.0 mm, 30.0-40.0 mm, 40.0-50.0 mm, 45.0 mm, 50.0-60.0 mm, 60.0-70.0 mm, 70.0-80.0 mm, 80.0-90.0 mm, 90.0-100.0 mm, or less than 100.0 mm. In one or more embodiments, the gasket 311 includes a second outer diameter 505 and a second inner diameter 507. In at least one embodiment, the second outer diameter 505 measures at least 5.0 mm, 5.0-50.0 mm, 5.0-10.0 mm, 10.0-15.0 mm, 15.0-20.0 mm, 20.0-25.0 mm, 23.0 mm, 25.0-30.0 mm, 30.0-35.0 mm, 35.0-40.0 mm, 40.0-45.0 mm, 45.0-50.0 mm, or less than 50.0 mm. In various embodiments, the second inner diameter 507 measures at least 5.0 mm, 5.0-50.0 mm, 5.0-10.0 mm, 10.0-15.0 mm, 15.0-20.0 mm, 20.0 mm, 20.0-25.0 mm, 25.0-30.0 mm, 30.0-35.0 mm, 35.0-40.0 mm, 40.0-45.0 mm, 45.0-50.0 mm, or less than 50.0 mm.

In one or more embodiments, the gasket 407 includes a first outer diameter 509 and a first inner diameter 511. In various embodiments, the first outer diameter 509 measures at least 5.0 mm, 5.0-100.0 mm, 5.0-10.0 mm, 10.0-20.0 mm, 20.0-30.0 mm, 30.0-40.0 mm, 40.0-50.0 mm, 45.0 mm, 50.0-60.0 mm, 60.0-70.0 mm, 70.0-80.0 mm, 80.0-90.0 mm, 90.0-100.0 mm, or less than 100.0 mm. In at least one embodiment, the first inner diameter 511 measures at least 5.0 mm, 5.0-100.0 mm, 5.0-10.0 mm, 10.0-20.0 mm, 20.0-30.0 mm, 30.0-40.0 mm, 40.0 mm, 40.0-50.0 mm, 50.0-60.0 mm, 60.0-70.0 mm, 70.0-80.0 mm, 80.0-90.0 mm, 90.0-100.0 mm, or less than 100.0 mm. In one or more embodiments, the gasket 407 includes a second outer diameter 513 and a second inner diameter 515. In at least one embodiment, the second outer diameter 513 measures at least 5.0 mm, 5.0-50.0 mm, 5.0-10.0 mm, 10.0-15.0 mm, 15.0-20.0 mm, 20.0-25.0 mm, 22.0 mm, 25.0-30.0 mm, 30.0-35.0 mm, 35.0-40.0 mm, 40.0-45.0 mm, 45.0-50.0 mm, or less than 50.0 mm. In various embodiments, the second inner diameter 513 measures at least 5.0 mm, 5.0-50.0 mm, 5.0-10.0 mm, 10.0-15.0 mm, 15.0-20.0 mm, 16.5 mm, 20.0-25.0 mm, 25.0-30.0 mm, 30.0-35.0 mm, 35.0-40.0 mm, 40.0-45.0 mm, 45.0-50.0 mm, or less than 50.0 mm.

In various embodiments, the interior tapered element 409 includes a first outer diameter 517 and a first inner diameter 519. In at least one embodiment, the first outer diameter 517 measures at least 5.0 mm, 5.0-50.0 mm, 5.0-10.0 mm, 10.0-15.0 mm, 15.0-20.0 mm, 20.0-25.0 mm, 22.0 mm, 25.0-30.0 mm, 30.0-35.0 mm, 35.0-40.0 mm, 40.0-45.0 mm, 45.0-50.0 mm, or less than 50.0 mm. In one or more embodiments, the first inner diameter 519 measures at least 5.0 mm, 5.0-50.0 mm, 5.0-10.0 mm, 10.0-15.0 mm, 15.0-20.0 mm, 20.0 mm, 20.0-25.0 mm, 25.0-30.0 mm, 30.0-35.0 mm, 35.0-40.0 mm, 40.0-45.0 mm, 45.0-50.0 mm, or less than 50.0 mm. In various embodiments, the interior tapered element 409 includes a second outer diameter 523 and a second inner diameter 525. In at least one embodiment, the second outer diameter 523 measures at least 1.0 mm, 1.0-20.0 mm, 1.0-2.0 mm, 2.0-4.0 mm, 4.5 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In one or more embodiments, the second inner diameter 525 measures at least 1.0 mm, 1.0-20.0 mm, 1.0-2.0 mm, 2.0-4.0 mm, 3.5 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm.

In one or more embodiments, the void 444 includes a diameter 527. In various embodiments, the diameter 527 measures at least 1.0 mm, 1.0-20.0 mm, 1.0-2.0 mm, 2.0-4.0 mm, 2.5 mm, 4.0-6.0 mm, 6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In some embodiments, the diameter 527 tapers between the end 431 and the chamber second end 408. For example, the diameter 527 measures 6.0 mm at the end 431 and measures 2.5 mm at the chamber second end 408.

In various embodiments, the shaft 415B of the bone mill 401 includes a diameter 530 that measures at least 1.0 mm, 1.0-20.0 mm, 1.0-2.0 mm, 2.0-4.0 mm, 4.0 mm, 4.0-6.0 mm, 6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In at least one embodiment, the shaft 415B includes a central region 531 that includes a second diameter 532. In one or more embodiments, the second diameter 532 measures at least 1.0 mm, 1.0-20.0 mm, 1.0-2.0 mm, 2.0-4.0 mm, 3.0 mm, 4.0-6.0 mm, 6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm.

FIG. 6 shows a cross section 600 of an additional embodiment of the impactor system 300 shown in FIG. 3. In one or more embodiments, the impactor system 300 includes a bone mill 601. In various embodiments, the bone mill 601 includes a plate 405, a shaft 603, and one or more blades 605. In at least one embodiment, the shaft 603 extends from the plate 405. The shaft 405 can be integrally formed with the plate 405 or attached to the plate 405 via any suitable means (e.g., fasteners, press fittings, snap fittings, bayonet fittings or luer locks, adhesives, welding, etc.). The blade 605 can be integrally formed with the shaft 603 or attached to the shaft 603 via any suitable means.

In one or more embodiments, the bone mill 401 is configured to rotatably couple to the plate 405. In various embodiments, the plate 405 includes a recess 604. In at least one embodiment, the recess 604 is configured to receive the second shaft 415B of the bone mill 401. In at least one embodiment, the bone mill 601 is rotatably coupled to the bone mill 401 via receipt of the second shaft 415B into the recess 604. In various embodiments, the bone mill 401 and bone mill 601 are rotably coupled via a gear system. For example, the second shaft 415B and plate 405 are linked via a gear system such that rotation of the second shaft 415B at a first rate causes corresponding rotation of the plate 405 at a second rate, less than, greater than, or equal to the first rate. In one or more embodiments, the gear system includes a gear ratio configured to provide a greater, lesser, or equal rate of rotation between the bone mill 401 and bone mill 601. The gear ratio can be any suitable ratio of rotation rate between the bone mill 401 and bone mill 601, including, but no limited to, 1:1, 1:2, 1:3, 1:4, 1:5, 1:6, 1:7, 1:8, 1:9, 1:10, or 2:1, 3:1, 4:1, 5:1, 6:1, 7:1, 8:1, 9:1, 10:1, or between 1:1 and 1:100, or between 1:1 and 100:1.

In various embodiments, the bone mill 401 and bone mill 601 are linked via a clutch mechanism (e.g., or a gear system linking the bone mill 401 and bone mill 601 includes a clutch mechanism). According to one embodiment, the clutch mechanism limits force transfer from the bone mill 401 to the bone mill 601 to a predetermined maximum force. In one example, the clutch mechanism transfers rotational force from the bone mill 401 to the bone mill 601. In this example, if the bone mill 401 rotates at a rate resulting in a rotational force in excess of a predetermined maximum force, the clutch mechanism experiences a slip such that the rotational force from the bone mill 401 is not translated to the bone mill 601. The clutch mechanism can include any suitable mechanism for conditionally de-coupling the bone mills 401, 601, such as, for example, magnetic torque transfer mechanisms, ball detent torque limitation mechanisms, sprag engagement mechanisms, and pawl detent mechanisms. In some embodiments, the bone mill 401 (e.g., or other bone mills described herein) includes a gear system and/or a clutch mechanism for rotationally coupling the bone mill 401 to a torque generation system.

In one or more embodiments, the recess 604 includes a diameter 606 that measures at least 1.0 mm, 1.0-20.0 mm, 1.0-2.0 mm, 2.0-4.0 mm, 4.0 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm.

In one or more embodiments, the shaft 603 and one or more blades 605 extend along the chamber 440 of the interior tapered element 409. In at least one embodiment, the tapered element 405 and/or the gasket 407 generally sheaths the bone mill 601. In some embodiments, while not shown in FIG. 6, a gasket 311 (see FIGS. 3, 4, 5) generally sheathes one or more of the gasket 407, bone mill 601, tapered element 405, or portions thereof. In various embodiments, during bone milling and bone graft generation processes, the interior tapered element 409, gasket 407, and/or gasket 311 remain static relative to rotation of the bone mills 401, 601.

In at least one embodiment, the interior tapered element 409 includes one or more mill structure 608A or 608B that extend into the chamber 440. In at least one embodiment, the bone mill 601 is configured to rotate bone material against the mill structures 608A, 608B to cut, crush, or pulverize bone. In various embodiments, the mill structures 608A, 608B include a helical pattern or other pattern based on the orientation and arrangement of the bone mill 601, or elements thereof, such as blade 605.

In one or more embodiments, the interior tapered element 409 is configured to attach to the gasket 407 via any suitable means including, but not limited to, fasteners, press fittings, snap fittings, bayonet fittings or luer locks, adhesives, and welding. In at least one embodiment, the tapered element includes slots 608A, 608B. In various embodiments, the gasket 407 includes prongs 609A, 609B. In various embodiments, the interior tapered element 409 is configured to attach to the gasket 407 by receiving the prongs 609A, 609B into the slots 608A, 608B. The interior tapered element 409 and gasket 407 can include any suitable number of slots and prongs, or other components, that may be mated to connect the interior tapered element 409 and gasket 407. In some embodiments, the gasket 407 includes the slots 608A, 608B and the interior tapered element 409 includes the prongs 609A, 609B.

In one or more embodiments, the plate 405 includes one or more voids 610 configured for passing bone material from the bone mill 401 to the bone mill 601 and/or interior tapered element 409. In various embodiments, the void 610 includes a circular shape or a circular sector shape. In at least one embodiment, the void 610 includes a width 613 between a first edge 611 and a second edge 612. In one or more embodiments, the width 613 measures at least 5.0 mm, 5.0-50.0 mm, 5.0-10.0 mm, 10.0-15.0 mm, 11.0 mm, 15.0-20.0 mm, 20.0-25.0 mm, 25.0 mm, 25.0-30.0 mm, 30.0-35.0 mm, 35.0-40.0 mm, 40.0-45.0 mm, 45.0-50.0 mm, or less than 50.0 mm.

In at least one embodiment, the plate 405 includes an outer diameter 615 and an inner diameter 617. In various embodiments, the outer diameter 615 measures at least 5.0 mm, 5.0-100.0 mm, 5.0-10.0 mm, 10.0-20.0 mm, 20.0-30.0 mm, 30.0-40.0 mm, 40.0 mm, 40.0-50.0 mm, 50.0-60.0 mm, 60.0-70.0 mm, 70.0-80.0 mm, 80.0-90.0 mm, 90.0-100.0 mm, or less than 100.0 mm. In one or more embodiments, the inner diameter 617 measures 5.0 mm, 5.0-100.0 mm, 5.0-10.0 mm, 10.0-20.0 mm, 20.0-30.0 mm, 30.0-40.0 mm, 38.0 mm, 40.0-50.0 mm, 50.0-60.0 mm, 60.0-70.0 mm, 70.0-80.0 mm, 80.0-90.0 mm, 90.0-100.0 mm, or less than 100.0 mm.

In at least one embodiment, the gasket 407 extends between a first end 616 and a second end 618. In various embodiments, at the first end 616, the gasket 407 shown in FIG. 6 includes a first outer diameter 619 and a first inner diameter 621. In one or more embodiments, the first outer diameter 619 measures at least 5.0 mm, 5.0-100.0 mm, 5.0-10.0 mm, 10.0-20.0 mm, 20.0-30.0 mm, 30.0-40.0 mm, 40.0-50.0 mm, 50.0 mm, 50.0-60.0 mm, 60.0-70.0 mm, 70.0-80.0 mm, 80.0-90.0 mm, 90.0-100.0 mm, or less than 100.0 mm. In at least one embodiment, the first inner diameter 621 measures at least 5.0 mm, 5.0-100.0 mm, 5.0-10.0 mm, 10.0-20.0 mm, 20.0-30.0 mm, 30.0-40.0 mm, 40.0-50.0 mm, 50.0 mm, 50.0-60.0 mm, 60.0-70.0 mm, 70.0-80.0 mm, 80.0-90.0 mm, 90.0-100.0 mm, or less than 100.0 mm.

In one or more embodiments, at the second end 618, the gasket 407 shown in FIG. 6 includes a second outer diameter 623 and a second inner diameter 625. In at least one embodiment, the second outer diameter 623 measures at least 5.0 mm, 5.0-50.0 mm, 5.0-10.0 mm, 10.0-15.0 mm, 15.0-20.0 mm, 20.0-25.0 mm, 25.0 mm, 25.0-30.0 mm, 30.0-35.0 mm, 35.0-40.0 mm, 40.0-45.0 mm, 45.0-50.0 mm, or less than 50.0 mm. In various embodiments, the second inner diameter 625 measures about at least 5.0 mm, 5.0-50.0 mm, 5.0-10.0 mm, 10.0-15.0 mm, 15.0-20.0 mm, 20.0 mm, 20.0-25.0 mm, 25.0-30.0 mm, 30.0-35.0 mm, 35.0-40.0 mm, 40.0-45.0 mm, 45.0-50.0 mm, or less than 50.0 mm.

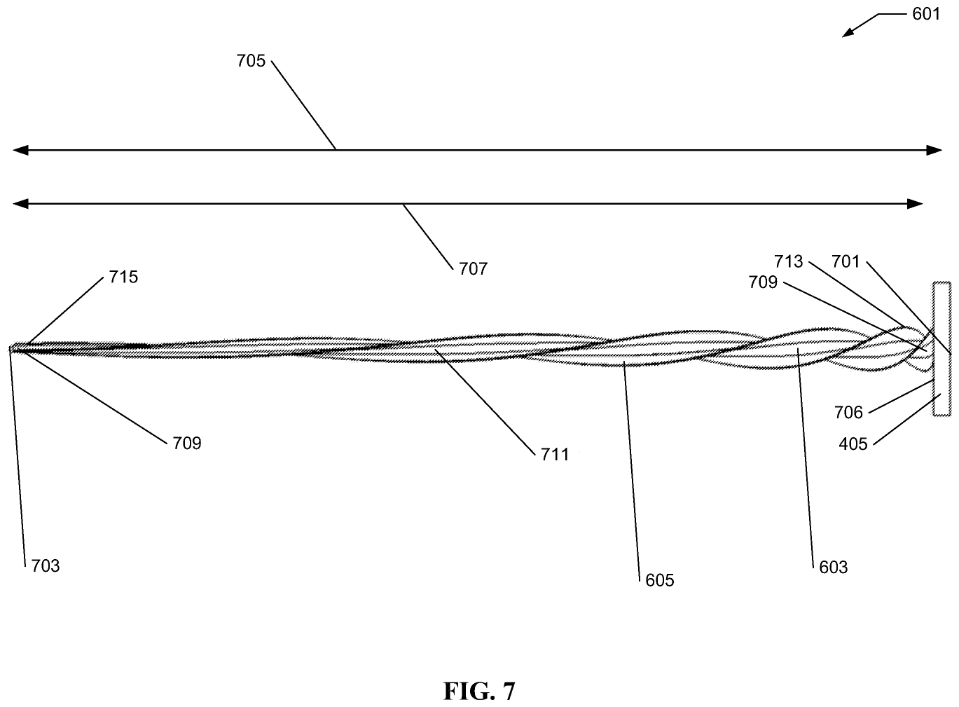

FIG. 7 shows an exemplary bone mill 601. In one or more embodiments, the bone mill 601 extends between a first end 701 and a second end 703. In at least one embodiment, a length 705 between the first end 701 and the second end 703 measures at least 100.0 mm, 100.0-500.0 mm, 100.0-150.0 mm, 150.0-200.0 mm, 200.0-250.0 mm, 250.0-300.0 mm, 280.0 mm, 300.0-350.0 mm, 313.0 mm, 350.0-400.0 mm, 400.0-450.0 mm, 450.0-500.0 mm, or less than 500.0 mm. In one or more embodiments, the shaft 603 and/or blade 605 include a length 707 between a surface 706 of the plate 405 and the second end 703. In various embodiments, the length 707 measures at least 100.0 mm, 100.0-500.0 mm, 100.0-150.0 mm, 150.0-200.0 mm, 200.0-250.0 mm, 250.0-300.0 mm, 274.0 mm, 300.0-350.0 mm, 313.0 mm, 350.0-400.0 mm, 400.0-450.0 mm, 450.0-500.0 mm, or less than 500.0 mm.

In at least one embodiment, the shaft 603 includes a first diameter 707. In various embodiments, the first diameter 707 measures at least 2.0 mm, 2.0-30.0 mm, 2.0-4.0 mm, 4.0-6.0 mm, 5.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, 20.0-22.0 mm, 22.0-24.0 mm, 24.0-26.0 mm, 26.0-28.0 mm, 28.0-30.0 mm, or less than 30.0 mm. In one or more embodiments, the shaft 603 includes a second diameter 709. In at least one embodiment, the second diameter 709 measures at least 0.5 mm, 0.5-20.0 mm, 0.5-1.0 mm, 1.0-2.0 mm, 2.0 mm, 2.0-4.0 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In various embodiments, the shaft 603 tapers from the first diameter 707 to the second diameter 709 along the length 707. For example, the shaft 603 includes the first diameter 707 towards the surface of the plate 405 and the shaft 603 tapers from the first diameter 707 to the second diameter 709 toward the second end 703. In one or more embodiments, a rate of change between the first diameter 707 and the second diameter 709 measures at least 0.1 mm/mm, 0.1-4.0 mm/mm, 0.1-0.5 mm/mm, 0.28 mm/mm, 0.5-1.0 mm/mm, 1.0-1.5 mm/mm, 1.5-2.0 mm/mm. 2.0-2.5 mm/mm, 2.5-3.0 mm/mm, 3.0-3.5 mm/mm. 3.5-4.0 mm/mm, or less than 4.0 mm/mm. In various embodiments, the shaft 103 includes a taper angle 711 toward the end 703. In at least one embodiment, the taper angle 711 measures at least 0.1 degrees, 0.1-30.0 degrees, 0.1-3.0 degrees, 0.627 degrees, 3.0-6.0 degrees, 6.0-9.0 degrees, 9.0-12.0 degrees, 12.0-15.0 degrees, 12.0-15.0 degrees, 15.0-18.0 degrees, 18.0-21.0 degrees, 21.0-24.0 degrees, 24.0-27.0 degrees, 27.0-30.0 degrees, or less than 30.0 degrees.

In various embodiments, the blade 605 includes a first pitch 713. In one or more embodiments, the first pitch 713 measures at least 1.0 mm, 1.0-20.0 mm, 1.0-2.0 mm, 2.0-4.0 mm, 4.0 mm, 4.0-6.0 mm, 6.0-8.0 mm, 8.0-10.0 mm, 10.0-12.0 mm, 12.0-14.0 mm, 14.0-16.0 mm, 16.0-18.0 mm, 18.0-20.0 mm, or less than 20.0 mm. In at least one embodiment, the blade 605 includes a second pitch 715. In various embodiments, the second pitch 715 measures at least 100.0 mm, 100.0-500.0 mm, 100.0-150.0 mm, 150.0-200.0 mm, 200.0-250.0 mm, 250.0-300.0 mm, 270.0 mm, 300.0-350.0 mm, 313.0 mm, 350.0-400.0 mm, 400.0-450.0 mm, 450.0-500.0 mm, or less than 500.0 mm. In one or more embodiments, the blade 605 tapers from the first pitch 713 to the second pitch 715 along the length 707. For example, the blade 605 includes the first pitch 713 towards the surface of the plate 405 and the blade 605 tapers from the first pitch 713 to the second pitch 715 toward the second end 703. In at least one embodiment, the rate of change from the first pitch 713 to the second pitch 715 measures at least 1.0 mm/mm, 1.0-50.0 mm/mm, 1.0-5.0 mm/mm, 5.0-10.0 mm/mm, 10.0-15.0 mm/mm, 15.0-20.0 mm/mm, 20.0-25.0 mm/mm, 25.0-30.0 mm/mm, 30.0-35.0 mm/mm, 35.0-40.0 mm/mm, 40.0-45.0 mm/mm, 45.0-50.0 mm/mm, or less than 50.0 mm/mm.

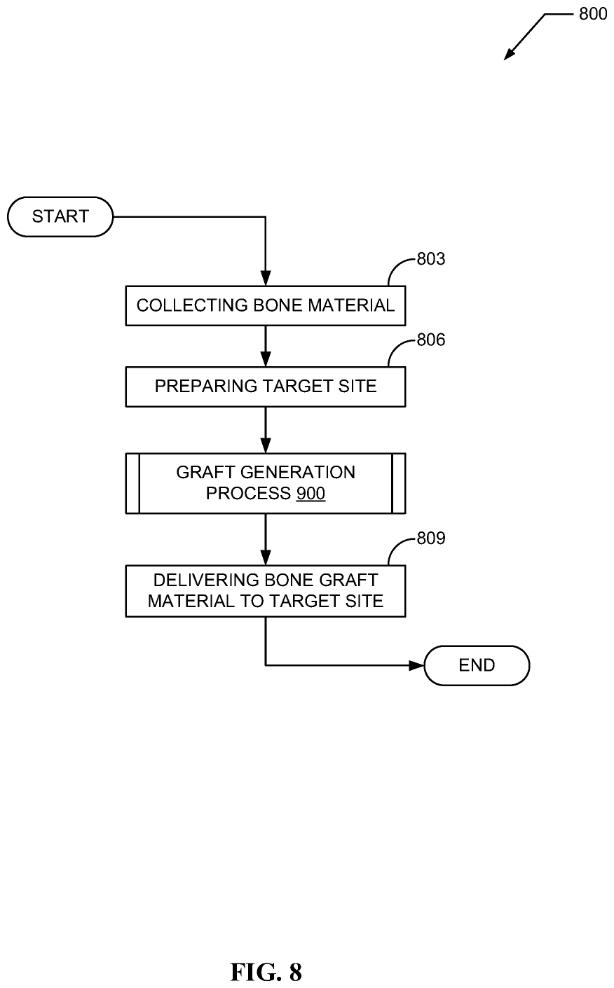

FIG. 8 shows an exemplary bone graft delivery process 800 that may be performed using an embodiment of the present impactor systems (e.g., the impactor system 100 shown in FIG. 1 or the impactor system 300 shown in FIG. 3). As will be understood by one having ordinary skill in the art, the steps and processes shown in FIG. 8 (and those of all other flowcharts and sequence diagrams shown and described herein) may operate concurrently and continuously, are generally asynchronous and independent, and are not necessarily performed in the order shown.

At step 803, the process 800 includes collecting bone material. The bone material can include, but is not limited to, autologous bone, allograft bone, alloplastic material, synthetic bone material, cadaveric bone material, and xenograft bone. In various embodiments, collecting the bone material includes extracting the bone material from the subject who will receive the bone material and/or a second subject, such as a human donor or animal. For example, a surgeon cuts bone material from a subject's iliac crest.

In at least one embodiment, collecting the bone material includes retrieving the bone material from a storage article (e.g., a container, a kit, or other suitable object for holding bone material). For example, a surgeon retrieves alloplastic material from a container. In some embodiments, collecting the bone material includes determining a quantity of bone material (e.g., mass, volume, etc.) required to perform the process 800. In at least one embodiment, determining the quantity of bone material includes measuring one or more dimensions of a target site (e.g., length, width, depth, volume, etc.) and estimating the quantity of material based on the one or more dimensions.

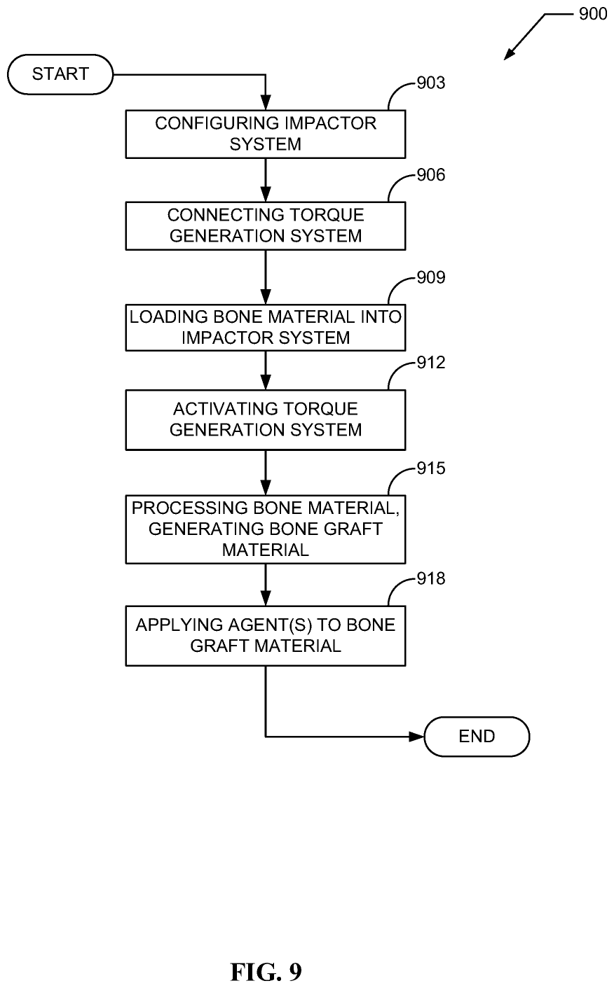

At step 806, the process 800 includes preparing a target site of a subject. In various embodiments, the impactor systems shown and described herein can be used in minimally invasive or open surgical procedures. In at least one embodiment, preparing the target site includes exposing bone. In one or more embodiments, preparing the target site includes reaming one or more bones, or segments thereof. In various embodiments, preparing the target site includes making one or more incisions into the subject. For example, a surgeon displaces or removes soft tissue at a target site to expose one or more regions of bone. In another example, a surgeon forms a narrow incision into a subject, inserts a first end of elongate tubing into the narrow incision, and guides the first end of the elongate tubing to a target site (e.g., a second end of the elongate tubing being connected to an impactor system). In one or more embodiments, preparing the target site includes scanning the subject via one or more imaging techniques and identifying the target site based on image data obtained therefrom. Non-limiting examples of imaging techniques include X-ray, magnetic resonance imaging (MRI), computed tomography (CT), ultrasound, and nuclear medicine imaging. In at least one embodiment, preparing the target site includes marking the subject (e.g., or a digital representation thereof) with indicia for indicating the target site. For example, a surgeon draws an outline of a target site onto a subject's skin. In another example, a computing device generates a virtual anatomy model based on image data of a subject. Continuing the example, the computing device receives or generates an indication of a target site on the virtual anatomy model. In the same example, the computing device estimates a volume of the target site and, based on the volume, determines a quantity of bone material required to fill the target site (e.g., fill completely or fill to a predetermined metric, such as 25%, 50%, or 75% of target site volume, or any other suitable metric). Continuing the example, the computing device generates and causes display of a notification that includes the quantity of bone material. In at least one embodiment, preparing the target site includes inserting one or more tracking markers (e.g., or arrays including a plurality of tracking markers) into the target site and/or along a navigation path to the target site (see step 903 shown in FIG. 9 and described herein).

In various embodiments, the process 800 includes performing one or more bone graft generation processes 900 (FIG. 9) to generate bone graft material (e.g., the bone graft material for delivery to the target site). In one or more embodiments, via the process 900, the bone material of step 803 is converted to bone graft material.

At step 809, the process 800 includes delivering the bone graft material to the target site. In various embodiments, delivering the bone graft material to the target site includes positioning an impactor system (e.g., the impactor system 100 or impactor system 300 shown in FIGS. 1 and 3, respectively). For example, a surgeon orients a tip 224 of the impactor system 100 to a target site. In some embodiments, the process 900 is performed subsequent to step 809 such that bone graft material is generated and delivered to a target site in a single stream operation. For example, a surgeon orients the bone impactor to a target site, loads bone material into the bone impactor, and activates a torque generation system connected to the bone impactor system. Continuing the example, in response to activation of the torque generation system, the bone impactor system mills the bone material into graft material and delivers the graft material to the target site virtually simultaneously.

In at least one embodiment, delivering the bone graft material to the target site includes determining a position of the impactor system and confirming that the impactor system is oriented to the target site based on the position. In various embodiments, step 809 includes generating image data of a target site via any suitable imaging technique. In at least one embodiment, step 809 includes analyzing the image data of the target site to a) identify the impactor system within the image data (e.g., or a component thereof, such as a tip), and b) determine that the impactor system is oriented to the target site. In one or more embodiments, the target site and/or impactor system include one or more tracking markers configured for detection within image data. In some embodiments, the impactor system includes one or more circuits configured to record and communicate a position of the impactor system (e.g., or readings that may be processed and analyzed to determine the same). In an exemplary scenario, delivering bone graft material to the target site includes, but is not limited to, a) providing at least one array releasably secured to an anatomical feature and at least one array releasably secured to an impactor system, b) providing a tracking system in communication with a navigation system (e.g., the tracking and navigation systems configured to captures images of the array and anatomical feature and the impactor system, respectively), c) navigating the impactor system (e.g., or tubing coupled to the same) to the anatomical feature while imaging the impactor system via the navigation system, and d) delivering the bone graft material from the impactor system to the anatomical feature while monitoring the anatomical feature via the tracking system.

Further description of exemplary target site and impactor system position monitoring is provided at step 903 described herein and shown in FIG. 9.

In various embodiments, step 809 includes covering and/or closing the target site following the delivery of the bone graft material. Target site covering and closure can be performed via any suitable means including, but not limited to, sutures, stiches, staples, tissue grafts, wound packing materials, wound wrapping materials, and adhesives.

FIG. 9 shows an exemplary graft generation process 900 that may be performed by an embodiment of the present impactor systems (e.g., the impactor system 100 shown in FIG. 1 or the impactor system 300 shown in FIG. 3).

At step 903, the process 900 includes configuring an impactor system. In some embodiments, configuring the impactor system includes selecting a particular impactor system or component(s) thereof, from a plurality of impactor systems or impactor system components. In at least one embodiment, the particular impactor system is selected based one or more aspects of the target site for which bone graft material is needed. In one example, a first target site includes a first depth and a second target site includes a second depth that exceeds the first depth. In this example, a first impactor system is configured for the first target site and a second impactor system is configured for the second target site, the second impactor system including a tapered element length that exceeds a tapered element length of the first impactor system. In another example, a first target site includes a first volume and a second impactor system includes a second volume that exceeds the first volume. Continuing the example, an impactor system is connected to a first hopper for use on the first target site and the impactor system is connected to a second hopper for use on the second target site, the second hopper including a volume that exceeds a volume of the first hopper. In at least one embodiment, the impactor system, or component(s) thereof, is selected based on image data from one or more scans and/or virtual rendering of the target site.

In some embodiments, the impactor system is used in conjunction with a tracking system configured to measure and record a position of the impactor system (e.g., absolute position or position relative to a target site). In one or more embodiments, the tracking system includes one or more tracking markers configured for insertion to the target site. In some embodiments, a plurality of tracking markers are configured into an array that is inserted to a target site. Non-limiting examples of tracking markers include, but are not limited to, radiopaque tags, signal-emitting circuits (e.g., near field communication sensors, light-emitting circuits, etc.), and circuits configured to generate and/or measure magnetic phenomena. In at least one embodiment, the tracking system includes one or more tracking markers that are attached to the impactor system. In some embodiments, configuring the impactor system includes attaching one or more tracking markers to the impactor system. According to one embodiment, configuring the impactor system includes inserting one or more tracking markers, or arrays containing the same, to the target site. In at least one embodiment, the tracking marker is releasably secured to one or more anatomical features at the target site. For example, configuring the impactor system includes securing an array of tracking markers to an anatomical feature, such as a vertebra. Continuing the example, a camera system tracks the array and a computing device renders a simulation of the anatomical feature and array on a display screen.

In one or more embodiments, the tracking system includes one or more computing devices configured to detect the tracking marker. Non-limiting examples of computing devices include cameras, optical tracking systems, and any suitable medical imaging systems. In various embodiments, the computing device is configured to detect the tracking marker by receiving signal from the tracking markers and/or by identifying the marker within image data associated with the target site. For example, the computing device is configured to analyze MRI scans of the target site and determine the position of one or more tracking markers based on the MRI scans. In at least one embodiment, the tracking system includes one or more sensors that are attached to the impactor system and configured to transmit data to the computing device. Non-limiting examples of sensors include camera sensors, magnetic position sensors, accelerometers, gyroscopes, and combinations of two or more sensors. In one example, a camera sensor is attached to a tip of a tapering element of the impactor system and the camera sensor is configured to transmit live image data to the computing device via any suitable wired or wireless means. In at least one embodiment, the computing device is configured to stream image data from the camera sensor to one or more displays and/or to one or more additional systems. For example, the computing device renders image data from the camera sensor onto one or more displays of an operating theater. In another example, the computing device streams the image data to a public or private webpage that displays the image data as a live surgical video feed. In at least one embodiment, the computing device stores image data and/or other sensor data at one or more data stores.

In one or more embodiments, the computing device is configured to generate a virtual rendering of a target site, one or more tracking markers, and/or the impactor system based on image data associated with a target site. In various embodiments, the computing device is configured to render the virtual rendering on a display. For example, the computing device analyze image data from one or more X-ray scans to determine a) a position of a target site in the image data, b) a position of one or more tracking markers, and/or c) a position of an impactor system. Continuing the example, the computing device generates and renders on a display a virtual rendering that includes the target site, the one or more tracking markers, and/or the impactor system.

In some embodiments, configuring the impactor system includes activating the tracking system, or one or more elements thereof. In at least one embodiment, activating the tracking system includes activating one or more sensors configured to measure a position of the impactor system (e.g., or a portion thereof, such a tip of a tapered element configured to pass bone graft material to a target site). In one or more embodiments, activating the sensor includes, but is not limited to, connecting the sensor to a power source, engaging a power setting of the sensor, or transmitting an activation command to the sensor via a computing device. In at least one embodiment, activating the sensor includes calibrating the sensor to record precise and accurate position of the impactor system (e.g., absolute position and/or position relative to the target site or one or more tracking markers).

In one or more embodiments, configuring the impactor system includes attaching hollow tubing to an end of the impactor system. For example, a surgeon secures hollow tubing to a tip 224 of the impactor system 100 shown in FIG. 2 and described herein.

Tubing can be secured to the impactor system via any suitable means including, but not limited to, fasteners, press fittings, snap fittings, bayonet fittings or luer locks, adhesives, and welding. In one example, a first end of elongate tubing is connected to the impactor system and a second end of the elongate tubing is positioned at a target site via a minimally invasive opening into the subject (e.g., a narrow incision, bore hole, etc.). In at least one embodiment, configuring the impactor system includes coupling a first end of tubing to a tip of the impactor system and coupling a second end of the tubing to an implant (e.g., the implant having been inserted to a target site within the subject).

At step 906, the process 900 includes connecting the impactor system to a torque generation system. In various embodiments, connecting the impactor system to the torque generation system includes inserting an end of the impactor system (e.g., or a component thereof) into a torque generation system and securing the end of the impactor system within the torque generation system. In one example, an end of a bone mill shaft of the impactor system is inserted into an electric drill and a chuck of the electric drill is engaged to secure the end of the bone mill shaft within the electric drill. In at least one embodiment, connecting the impactor system to the torque generation system includes placing and/or securing a sterile cover over the torque generation system.

At step 909, the process 900 includes loading bone material into the impactor system. In at least one embodiment, loading bone material into the impactor system includes inserting bone material into one or more hoppers of the impactor system. For example, bone extracted from a subject is inserted into a hopper 103 of an impactor system 100 (see FIG. 1). In various embodiments, loading the bone material into impactor system includes placing and/or securing a sterile cover over the hopper.

At step 912, the process 900 includes activating the torque generation system. In at least one embodiment, activating the torque generation system includes, but is not limited to, connecting the sensor to a power source, providing an activation input to the torque generation system, or transmitting an activation command to the sensor via a computing device. In one example, the torque generation system is an electric drill and activating the electric drill includes depressing a trigger mechanism.

At step 915, the process 900 includes processing the bone material into bone graft material via the impactor system. In one or more embodiments, processing the bone material includes, but is not limited to, a) milling the bone material via one or more bone mills, b) cutting the bone material via one or more cutting elements, and/or c) filtering the bone material via one or more size filters. In at least one embodiment, step 915 of the process 900 may be performed virtually simultaneously to delivery of the resultant bone graft material to the target site (e.g., step 809 described herein and shown in FIG. 8).

At step 918, the process 900 includes applying, via the impactor system, one or more agents to the bone material and/or bone graft material. In at least one embodiment, the impactor system includes one or more means for introducing or applying one or more agents to bone material and/or bone graft material. Non-limiting examples of agent introduction means include agent dispensers (e.g., pumps, reservoirs, nozzles, brushes, etc.) and components subjected to doping or coating with one or more agents. In some embodiments, the agent introduction means automatically engage upon activation of the torque generation system and/or upon bone material passing within a proximity of the agent introduction means. In at least one embodiment, the agent introduction means are passive such that agent is introduced to bone material upon contact with the agent introduction means. In one or more embodiments, the impactor system includes one or more controls (e.g., buttons, triggers, switches, etc.) that can be engaged by a user via manual input or command from a computing device. In at least one embodiment, one or more agents are introduced to the bone material prior to or during insertion of the bone material into the impactor system.

From the foregoing, it will be understood that various aspects of the processes described herein are software processes that execute on computer systems that form parts of the system. Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a general purpose computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such connection is properly termed and considered a computer-readable medium. Combinations of the above should also be included within the scope of computer-readable media. Computer-executable instructions comprise, for example, instructions and data which cause a general purpose computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed systems may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed system are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the systems are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the system is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed systems will be readily discernible from the description herein, by those of ordinary skill in the art. Many embodiments and adaptations of the disclosure and claimed systems other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

Aspects, features, and benefits of the claimed devices and methods for using the same will become apparent from the information disclosed in the exhibits and the other applications as incorporated by reference. Variations and modifications to the disclosed systems and methods may be effected without departing from the spirit and scope of the novel concepts of the disclosure.

It will, nevertheless, be understood that no limitation of the scope of the disclosure is intended by the information disclosed in the exhibits or the applications incorporated by reference; any alterations and further modifications of the described or illustrated embodiments, and any further applications of the principles of the disclosure as illustrated therein are contemplated as would normally occur to one skilled in the art to which the disclosure relates.

The foregoing description of the exemplary embodiments has been presented only for the purposes of illustration and description and is not intended to be exhaustive or to limit the devices and methods for using the same to the precise forms disclosed. Many modifications and variations are possible in light of the above teaching.

The embodiments were chosen and described in order to explain the principles of the devices and methods for using the same and their practical application so as to enable others skilled in the art to utilize the devices and methods for using the same and various embodiments and with various modifications as are suited to the particular use contemplated. Alternative embodiments will become apparent to those skilled in the art to which the present devices and methods for using the same pertain without departing from their spirit and scope. Accordingly, the scope of the present devices and methods for using the same is defined by the appended claims rather than the foregoing description and the exemplary embodiments described therein.

What is claimed is:

1. A bone impactor system, comprising:
at least one hopper; and
a sterile compartment configured to receive bone material from the at least one hopper, wherein the sterile compartment comprises:
a first end;
a second end opposite the first end; and
between the first end and the second end:
at least one bone milling mechanism configured to:
rotatably couple to a torque generation system; and
upon activation of the torque generation system, mill the bone material; and
at least one rotary cutting mechanism rotatably coupled to the at least one bone milling mechanism and configured to, upon activation of the torque generation system, cut the bone material, wherein milling and cutting the bone material transforms the bone material into bone graft material; and
an opening at the second end wherein the bone graft material exits the sterile compartment.

2. The bone impactor system of claim 1, wherein the sterile compartment further comprises, between the first end and a second end, at least size one filter configured to selectively filter the bone graft material based on a predetermined pore size.

3. The bone impactor system of claim 2, wherein the at least one bone milling mechanism, the at least one rotary cutting mechanism, and the at least one size filter are centrally aligned along a longitudinal axis extending from the first end to the second end.

4. The bone impactor system of claim 3, wherein the sterile compartment further comprises at least one tapered element between the first end and the second end, wherein the at least one tapered element defines the opening at the second end and is configured to:
receive the bone graft material from the at least one size filter; and
concentrate the bone graft material toward the opening at the second end.

5. The bone impactor system of claim 4, wherein:
the at least one size filter is rotatable coupled to the at least one bone milling mechanism or the at least one cutting mechanism; and
the bone impactor system further comprises at least one secondary bone milling mechanism rotatably coupled to the at least one size filter and configured to mill the bone graft material upon activation of the torque generation system.

6. The bone impactor system of claim 5, wherein the at least one secondary bone milling mechanism is further configured to, upon activation of the torque generation system, drive the bone graft material through the at least one tapered element and out the opening at the second end.

7. The bone impactor system of claim 6, wherein the at least one secondary bone milling mechanism is integrally formed with the at least one tapered element.

8. The bone impactor system of claim 7, wherein the at least one secondary bone milling mechanism comprises a plurality of helical structures that extend along at least a portion of the at least one tapered element.

9. The bone impactor system of claim 6, wherein the at least one secondary bone milling mechanism is configured to rotate independently from the at least one tapered element.

10. The bone impactor system of claim 4, further comprising a compression sleeve configured to attach to the sterile compartment, wherein a portion of the at least one tapered element extends through the compression sleeve.

11. The bone impactor system of claim 1, further comprising:
at least one array configured to be releasably secured to a target site, wherein:
the at least one array comprises a plurality of tracking markers;
the plurality of tracking markers are radiopaque; and
the sterile compartment comprises at least one additional tracking marker at the second end;
an imaging system configured to generate image data corresponding to the at least one array and the at least one additional tracking marker; and
a computing device connected to a display and configured to:
receive the image data from the imaging system;
generate a simulation of the target site, the at least one array, and at least the second end based on the image data; and
cause the display to render the simulation.

12. The bone impactor system of claim 1, further comprising a coupling mechanism configured to couple the second opening to an implant.

13. A bone impactor system, comprising:
at least one hopper configured to hold bone material; and
a compartment, wherein the compartment comprises:
a first opening configured to connect the at least one hopper to the compartment;
at least one milling mechanism configured to:
receive, via the first opening, the bone material from the at least one hopper; and
mill the bone material into a first set of bone graft material;
at least one rotary cutting mechanism configured to:
receive the first set of bone graft material; and
cut the first set of bone graft material into a second set of bone graft material;
at least one size filter configured to:
receive the second set of bone graft material; and
selectively filter the second set of bone graft material into a third set of bone graft material; and
at least one tapered element defining a second opening of the compartment and configured to:
receive the third set of bone graft material; and
direct the third set of bone graft material out of the compartment via the second opening.

14. The bone impactor system of claim 13, wherein the at least one rotary cutting mechanism is configured to receive the bone material from the at least one milling mechanism.

15. The bone impactor system of claim 13, wherein the at least one bone milling mechanism comprises an auger.

16. The bone impactor system of claim 13, wherein the at least one rotary cutting mechanism comprises at least two blades.

17. The bone impactor system of claim 13, wherein the at least one hopper is a second sterile compartment.

18. The bone impactor system of claim 13, further comprising:
a computing device configured to:
track a position of the at least one tapered element relative to a target site based on image data from an imaging system; and
command a display to render a simulation of the position of the at least one tapered element relative to the target site;
the imaging system configured to generate the image data via imaging at least one tracking marker and at least one additional tracking marker;
the at least one tracking marker affixed to the at least one tapered element; and
the at least one additional tracking marker releasably secured to the target site.

19. The bone impactor system of claim 13, further comprising:
tubing comprising a first end and a second end opposite the first end; and
a coupling mechanism configured to:
couple the first end of the tubing to the second opening; and
couple the second end of the tubing to an implant, wherein the tubing is configured to:
receive the third set of bone graft material via the second opening; and
deliver the third set of bone graft material into the implant.

20. A method for preparing and delivering a bone graft, comprising:
inserting bone material into a bone impactor device;
processing, via the bone impactor device, the bone material into bone graft material, wherein processing the bone material comprises:
milling the bone material via a first rotational element of the bone impactor device;
cutting the bone material via a second rotational element of the bone impactor device; and
filtering the bone material to isolate the bone graft material; and
delivering, via the bone impactor device, the bone graft material to a target site.

21. The method of claim 20, further comprising introducing, via the bone impactor device, at least one agent to the bone graft material prior to delivering the bone graft material.

22. The method of claim 20, further comprising monitoring, via a sensor of the bone impactor device, a position of the bone impactor device during delivery of the bone graft material.

23. The method of claim 20, further comprising selecting the bone impactor device from a kit based on the target site of the subject, wherein the kit comprises two or more bone impactor devices.

24. The method of claim 20, further comprising connecting the bone impactor device to a torque generation system.

25. The method of claim 20, further comprising:
releasably securing at least one array to the target site, wherein the at least one array comprises radiopaque material;
generating, via an imaging system, image data of the at least one array;
receiving the image data at a computing device;
tracking, via the computing device, the delivery of the bone graft material to the target site, wherein tracking the delivery comprises:
generating a simulation of the target site based on the image data; and
rendering the simulation on a display connected to the computing device.

26. The method of claim 20, wherein the target site comprises an implant and the method further comprises:
coupling the bone impactor device to the implant; and
delivering the bone graft material into the implant via the bone impactor device.
